SEQUENCE LISTING (20 sequences)

SEQ ID NO: 1 - protein, 560 aa, Pseudomonas syringae
AADPALLCTSVDLMSTSEHQQLATFNDTAHPYPRDVLIHQLIEQQAAQRPDACAVRGDSGFLLTYAELNQQANQLAHRLIELGVEPDTRVAVSLRRGAEMVVALLGILKAGGAYVPIDPDLPSARQAYMLEDSSPQAVLTTRDLSDNLPASDLPVLVLDGHDDRAQLARQQSVNPDAKALGLQPNHLAYVLYTSGSTGTPKGVMNEHLGVVNRLLWARDAYQVNSQDRVLQKTPFGFDVSVWEFFLPLLAGAELVNAPPGGHQDPDYLAQVMSGAGITLLHFVPSMLDVFLHERSTRDFPQLRRVLCSGEALPRALQRRFEQQLKGVELHNLYGPTEAAIDVTAMECRPTDPGDSVPIGRPIANIQIHVLDALGQLQPMGVAGELHIGGIGVARGYLNQPQLSAERFIADPFSNDPQARLYKTGDVGRWLANGALEYLGRNDFQVKIRGLRIEIGEIEAALAKHPAVREAVVTAREDIFGDKRLVAYYTQSAEHTAVDLEALRSHLQQVLPEYMVPAIYVLLEAMPLTSNGKLDRKALPAPELKAQAPGRAPKAGSETII

SEQ ID NO: 2 - protein, 560 aa, Pseudomonas syringae
AADPALLCTSVDLMSTSEHQQLATFNDTAHPYPRDVLIHQLIEQQAAQRPDACAVRGDSGFLLTYAELNQQANQLAHRLIELDVEPDTRVAVSLRRGAEMVVALLGILKAGGAYVPIDPDLPSARQAYMLEDSSPQAVLTTRDLSDNLPASDLPVLVLDGHDDRAQLARQQSVNPDAKALGLQPNHLAYVLYTSGSTGTPKGVMNEHLGVVNRLLWARDAYQVNSQDRVLQKTPFGFDVSVWEFFLPLLAGAELVNALPGGHQDPDYLAQVMSDAGITLLHFVPSMLDVFLHHRSTRDFPQLRRVLCSGEALPRALQRRFEQQLKGVELHNLYGPTEAAIDVTAMECRPTDPGDSVPIGRPIANIQIHVLDALGQLQPMGVAGELHIGGIGVARGYLNQPQLSAERFIADPFSNDPQARLYKTGDVGRWLANGALEYLGRNDFQVKIRGLRIEIGEIEAALAKHPAVHEAVVTAREDIFGDKRLVAYYTQSAEHTAVDLEALRSHLQQVLPEYMVPAIYVLLEAMPLTSNGKLDRKALPAPELKAQAPGRAPKAGSETII

SEQ ID NO: 3 - protein, 560 aa, Pseudomonas syringae
AADPALLCTSVDLMSTSEHQQLATFNDTAHPYPRDVLIHQLIEQQAAQRPDACAVRGDSGFLLTYAELNQQANQLAHRLIELDVEPDTRVAVSLRRGAEMVVALLGILKAGGAYVPIDPDLPSARQAYMLEDSSPQAVLTTRDLSDNLPASDLPVLVLDGHDDRAQLARQQSVNPDAKALGLQPNHLAYVLYTSGSTGTPKGVMNEHLGVVNRLLWARDAYQVNSQDRVLQKTPFGFDVSVWEFFLPLLAGAELVNAPPGGHQDPDYLAQVMSDAGITLLHFVPSMLDVFLHHRSTRDFPQLRRVLCSGEALPRALQRRFEQQLKGVELHNLYGPTEAAIDVTAMECRPTDPGDSVPIGRPIANIQIHVLDALGQLQPMGVAGELHIGGIGVARGYLNQPQLSAERFIADPFSNDPQARLYKTGDVGRWLANGALEYLGRNDFQVKIRGLRIEIGEIEAALAKHPAVHEAVVTAREDIFGDKRLVAYYTQSAEHTAVDLEALRSHLQQVLPEYMVPAIYVLLEAMPLTSNGKLDRKALPAPELKAQAPGRAPKAGSETII

SEQ ID NO: 4 - protein, 560 aa, Pseudomonas syringae
AADPALLCTSVDLMSTSEHQQLATFNDTAHPYPRDVLIHQLIEQQAAQRPDACAVRGDSGFLLTYAELNQQANQLAHRLIELGVEPDTRVAVSLRRGAEMVVALLGILKAGGAYVPIDPDLPSARQAYMLEDSSPQAVLTTRDLSDNLPASDLPVLVLDGHDDRAQLARQQSVNPDAKALGLQPNHLAYVLYTSGSTGTPKGVMNEHLGVVNRLLWARDAYQVNSQDRVLQKTPFGFDVSVWEFFLPLLTGAELVNAPPGGHQDPDYLAQVMSDAGITLLHFVPSMLDVFLHHRSTRDFPQLRRVLCSGEALPRALQRRFEQQLKGVELHNLYGPTEAAIDVTAMECRPTDPGDSVPIGRPIANIQMHVLDALGQLQPMGVAGELHIGGIGVARGYLNQPQLSAERFIADPFSNDPQARLYKTGDVGRWLANGALEYLGRNDFQVKIRGLRIEIGEIEAALAKHPAVHEAVVTAREDIFGDKRLVAYYTQSAEHTAVDLEALRSHLQQVLPEYMVPAIYVLLEAMPLTSNGKLDRKALPAPELKAQAPGRAPKAGSETII

SEQ ID NO: 5 - protein, 15 aa, Escherichia coli
IDYGLDSVRMMALAA

SEQ ID NO: 6 - protein, 15 aa, Streptomyces
LDLGLDSLAVYEVVT

SEQ ID NO: 7 - protein, 15 aa, Streptomyces
TDLGYDSLTVYEIVT

SEQ ID NO: 8 - protein, 15 aa, Streptomyces
TELGYDSLALMETAA

SEQ ID NO: 9 - protein, 15 aa, Streptomyces
VDLGYDSLALLETAA

SEQ ID NO: 10 - protein, 69 aa, Escherichia coli
SKAELREVILPLLDESDEPFDDDNLIDYGLDSVRMMALAARWRKVHGDIDFVMLAKNPTIDAWWKLLSR

SEQ ID NO: 11 - protein, 58 aa, Brevibacillus brevis
MPVTEAQYVAPTNAVESKLAEIWERVLGVSGIGILDNFFQIGGHSLKAMAVAAQVHRE

SEQ ID NO: 12 - protein, 60 aa, Escherichia coli
LPELKAQAPGRAPKAGSETIIAAAFSSLLGCDVQDADADFFALGGHSLLAMKLAAQLSRQ

SEQ ID NO: 13 - protein, 27 aa, Brevibacillus brevis
YQVELPLKVLFAQPTIKALAQYVATRS

SEQ ID NO: 14 - protein, 28 aa, Escherichia coli
VARQVTPGQVMVASTVAKLATIIDAEED

SEQ ID NO: 15 - DNA, 69 bp, Artificial Sequence
  Primer
ccagcacctatccccgcckccrmarmagmactgsstgmagytatcsytscattgctggacgagtccgat

SEQ ID NO: 16 - DNA, 63 bp, Artificial Sequence
  Primer
gaggtgatcctgccgsytctggmtgmakccgmtgmascakytgmtgmtgacaacctgatcgac

SEQ ID NO: 17 - DNA, 60 bp, Artificial sequence
  Primer
gaacccttcgatgacgmtrmcsytrytgmtkmtgstsytgmttcggtgcgcatgatggcg

SEQ ID NO: 18 - DNA, 60 bp, Artificial Sequence
  Primer
gcgcttggcggtcattcgsytsytgcarygrmactggcasmacagttaagtcggcaggtt

SEQ ID NO: 19 - DNA, 51 bp, Artificial Sequence
  Primer
cgccaggtgacgccggggsmagytryggytsmatcaactgtcgccaaactg

SEQ ID NO: 20 - DNA, 57 bp, Artificial Sequence
  Primer
cagttaagtcggcaggttgcasstsmagytrctscagstcaagtgatggtcgcgtca

(19) United States
(12) Patent Application Publication
(10) Pub. No.: US 2010/0048422 A1
(43) Pub. Date: Feb. 25, 2010

(54) Methods of Producing Modified Assembly Lines and Related Compositions

(76) Inventors: Christopher T. Walsh, Wellesley, MA (US); Michael A. Fischbach, Boston, MA (US); Jonathan R. Lai, Roxbury, MA (US); David R. Liu, Lexington, MA (US); Zhe Zhou, Boston, MA (US)

Correspondence Address: CLARK & ELBING LLP, 101 FEDERAL STREET, BOSTON, MA, 02110, US

(21) Appl. No.: 11/989,257

(22) PCT Filed: Jul. 21, 2006
(86) PCT No.: PCT/US06/28487

§ 371 Date: Oct. 14, 2009

Related U.S. Application Data
(60) Provisional application No. 60/701,807, filed on Jul. 21, 2005.

(51) Int. Cl. C40B 40/06; C12Q 1/02; C12Q 1/18; C12N 1/21; C12N 1/19

(52) U.S. Cl. 506/16; 435/29; 435/32; 435/252.3; 435/254.2

(57) ABSTRACT

The present invention provides a method producing a modified assembly line, such as those that produce non-ribosomal peptides and polyketides. The modified assembly lines of the invention can be used to produce novel compounds with therapeutic activities. The invention also provides organisms containing modified assembly lines and libraries of modified assembly lines.

### BACKGROUND OF THE INVENTION

Non-ribosomal peptides (NRPs) and polyketides (PKs) are classes of secondary metabolites produced in a variety of organisms. Many members from this classification of natural products exhibit medicinally relevant properties including antimicrobial (e.g., vancomycin and erythromycin), antitumor (e.g., bleomycin and epothilone), antifungal (e.g., soraphen and fengycin), immunosuppressant (e.g., cyclophilin and rapamycin) and cholesterol-lowering (e.g., lovastatin) activity.

Although NRP and PK natural products are chemically diverse, these types of compounds are biosynthesized in their cognate producer organisms in a similar manner by multienzymatic megacomplexes known as non-ribosomal peptide synthetases and polyketide synthases. These large proteins construct the framework of NRPs and PKs in an assembly-line fashion from simple chemical monomers (amino acids in the case of NRPSs, and acyl-CoA thioesters in the case of PKSs). For more information on classification of NRPs and PKs, see Cane D E, Walsh C T, and Khosla C, Science, 1998, 282, 63 and references therein.

The power of NRPs and PKs as potential drugs lies in their diverse and complicated chemical structures. Generally, it is the intricacy of these natural products that makes them (or variants thereof) difficult to access synthetically. Several examples exist where laborious synthetic routes have been developed, rarely successfully, for NRPs or PKs. Additionally, various moieties on such molecules are inaccessible to modification by organic synthesis, or can only be produced at low yields using such techniques. This difficultly in synthesis and modification of the NRP and PK natural products underscores the need for alternative strategies to enhance synthesis and create variants of these molecules.

Despite the apparent modular structure of the NRPSs, it has, prior to present invention, in practice been difficult to swap domains so that the resulting NRPS is active. Substitution of one domain for another generally yields great (e.g., >10-fold) reductions in yield (see FIG. 8; Eppelmann et al., Biochemistry (2002) 41, 9718; and FIG. 9; McDaniel et al., Proc Natl Acad Sci USA 96, 1846-1841, 1999) and results in increase in production of undesirable biosynthetic side products. These changes may be a result of disruptions of inter-domain quaternary interactions. Previously, it had been concluded that NRPSs are not modular, and that domain swapping requires great knowledge of the specific NRPS quaternary structure of the protein to be modified.

Thus, there is a need for new methods to produce novel varieties NRPs and PKs and a need for methods that increase the yields of such NRPs and PKs.

### SUMMARY OF THE INVENTION

In a first aspect, the invention provides a method of generating a modified assembly line which includes the steps of (a) providing a first gene encoding a polypeptide, wherein the polypeptide includes at least one (e.g., at least 2, 3, 4, 5, 7, 10, 15, 20, 25, 30) domain of a first assembly line (e.g., an NRPS, PKS, or NRPS-PKS hybrid); (b) creating at least 15 (e.g., at least 20, 25, 30, 40, 50, 60, 75, 100, 200, 500, 750, 1000, 5000, 7500, 10,000, 25,000, 50,000, 100,000, 500,000, 1,000,000, 10,000,000, 100,000,000, 1,000,000,000) unique mutations in the nucleic acid encoding the domain, thereby creating unique variants; (c) introducing the unique mutations into second genes (e.g., genes derived from the same biosynthetic gene as the first gene) encoding at least one domain of a second assembly line (e.g., an assembly line derived from the same or different biosynthetic gene or genes as the first assembly line); (d) expressing the second assembly line in a cell, for example a bacterium (e.g., Bacillus subtilis, Pseudomonas syringae, Streptomyces sp., or Esherichia coli) or a fungal cell (e.g., a yeast cell); and (e) identifying a variant generated from step (b); using the cell in a selection or screen, wherein the selection or screen identifies a modified assembly line that alters the amount or structure of a product (e.g., an antibiotic, antifungal, antineoplastic agent, or immunosupressant) of the second assembly line; The method may further include repeating steps (b) through (e) at least once (e.g., at least 2, 3, 4, 5, 6, 7, 10, 15, 20, 35, 30, 50, 75, 100 times). The method may also include replacing at least one domain in the second assembly line with a domain from a third assembly line (e.g., an assembly line derived from the same biosynthetic assembly line as the first assembly line) prior to the identifying step (e). The method may include a creating step (b) which further includes modifying a second domain of the polypeptide coded for by the first gene. The creating step (b) may be performed in vitro. The creating step (b) may be performed by random mutagenesis (e.g., error prone PCR). The introducing step (c) may include replacing at least one domain of the second gene with the variant. The selection or screen may be performed by observing antibacterial or antifungal activity of the product. The selection may be performed on solid media or may be performed in liquid media. The second assembly line may be an NRPS, a PKS, or an NRPS-PKS hybrid. The polypeptide may include all domains of the assembly line.

In another aspect the invention also provides an organism including a modified assembly line of the first aspect.

In another aspect, the invention provides a library produced by the method including steps (a)-(c) of the first aspect to produce a library including at least 15 (e.g., at least 20, 25, 30, 40, 50, 60, 75, 100, 200, 500, 750, 1000, 5000, 7500, 10,000, 25,000, 50,000, 100,000, 500,000, 1,000,000, 10,000,000, 100,000,000, 1,000,000,000) nucleic acids encoding unique variants.

By “assembly line” is meant a polypeptide or plurality of interacting polypeptides that form multimodular enzymes which synthesize one or more of the following categories of small molecules: (i) nonribosomal peptides, (ii) polyketides, and (iii) nonribosomal peptide-polyketide hybrids. Assembly lines comprise an initiation module and a termination module. Assembly lines may further comprise one, two, three, four, five, six, seven, or more elongation modules. Assembly lines may be synthases, synthetases, or a combination thereof.

By “module” is meant a set of domains. A plurality of modules comprise an assembly line (e.g., an NRPS or PKS). One or more polypeptides may comprise a module. Combinations of modules can catalyze a series of reactions to form larger molecules. In one example, a module may comprise a C (condensation) domain, an A (adenylation) domain, and a peptidyl carrier protein domain.

By “initiation module” is meant a module which is capable of providing a monomer to a second module (e.g., an elongation or termination module). In the case of an NRPS, an initiation module comprises, for example, an A (adenylation) domain and a PCP (peptidyl carrier protein) (e.g., a T (thiolation)) domain. The initiation module may also contain an E (epimerization) domain. In the case of a PKS, the initiation module comprises an AT (acetyltransferase) domain and an acyl carrier protein (ACP) domain. Initiation modules are preferably at the amino terminus of a polypeptide of the first module of an assembly line, and each assembly line preferably contains one initiation module.

By “elongation module” is meant a module which adds a monomer to another monomer or to a polymer. An elongation module may comprise a C (condensation), Cy (heterocyclization), E, MT (methyltransferase), Ox (oxidase), or Re (reductase) domain; an A domain; or a T domain. An elongation domain may further comprise additional E, Re, DH (dehydration), MT, NMet (N-methylation), or Cy domains.

By “termination module” is meant a module that releases the molecule (e.g., an NRP, PK, or combination thereof) from the assembly line. The molecule may be released by, for example, hydrolysis or cyclization. Termination modules may comprise a TE (thioesterase), C, or Re domain. The termination module is preferably at the carboxy terminus of a polypeptide of an NRPS or PKS. The termination module may further comprise additional enzymatic activities (e.g., oligomerase activity).

By “domain” is meant a polypeptide sequence, or a fragment of a larger polypeptide sequence, with a single enzymatic activity. Thus, a single polypeptide may comprise multiple domains. Multiple domains may form modules. Examples of domains include C (condensation), Cy (heterocyclization), A (adenylation), T (thiolation), TE (thioesterase), E (epimerization), MT (methyltransferase), Ox (oxidase), Re (reductase), KS (ketosynthase), AT (acyltransferase), KR (ketoreductase), DH (dehydratase), and ER (enoylreductase).

By “nonribsomally synthesized peptide,” “nonribosomal peptide,” or “NRP” is meant any polypeptide not produced by a ribosome. NRPs may contain cyclized or branched amino acids, or any combination thereof. NRPs include peptides produced by an assembly line.

By “polyketide” is meant a compound comprising multiple ketyl units.

By “nonribosomal peptide synthetase” is meant a polypeptide or series of interactaing polypetide that produce a nonribosomal peptide.

By “polyketide synthase” is meant a polypeptide or series of polypeptides that produce a polyketide.

By “alter an amount” is meant to change the amount, by either increasing or decreasing. An increase or decrease maybe by 3%, 5%, 8%, 10%, 15%, 20%, 30%, 40%, 50%, 60%, 70%, 80%, 90%, 100%, or more.

By “alter a structure” is mean any change in a chemical (e.g., covalent or noncovanlent) bond as compared to a reference structure.

By “mutation” is meant an alteration in the nucleic acid sequence such that the amino acid sequence encoded by the nucleic acid sequence has at least one amino acid alteration from a naturally occurring sequence. The mutation may, without limitation, be an insertion, deletion, frameshift mutation, or a missense mutation. This term also describes a protein encoded by the mutant nucleic acid sequence.

By “variant” is meant a polypeptide or polynucleotide with at least 10%, 20%, 30%, 40%, 50%, 60%, 70%, 80%, 90%, 95%, or 99% sequence identity to a reference sequence.

Sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e⁻³ and e⁻¹⁰⁰ indicating a closely related sequence.

Other features and advantages of the invention will be apparent from the following Detailed Description, the drawings, and the claims.

### BRIEF DESCRIPTION OF THE DRAWINGS

FIG. 1 is a chemical structure showing the chemical features of nonribosomal peptides.

FIG. 2 is a set of chemical structures of exemplary NRPs, PKS, and NRP/PK hybrids.

FIG. 3 is a schematic diagram of an NRPS assembly line.

FIG. 4 is a schematic diagram of NRPS adenylation and peptidyl carrier protein.

FIG. 5 is a schematic diagram showing the NRPS condensation domain.

FIG. 6 is a schematic diagram showing termination by the thioesterase (TE) domain.

FIG. 7 is a schematic diagram showing colinearity and modularity of the NRPS that produces tyrocidine.

FIG. 8 is a set of mass spectroscopy traces showing that replacement of Asp domain with an Asn domain results is a substantial reduction in assembly line production.

FIG. 9 is a table showing yield reduction caused by replacement of domains in the PKS that produces 6dEB.

FIG. 10 is a schematic diagram showing similar gene/module organization among three NRPSs.

FIG. 11 is a phylogenetic tree showing similarities between NRPSs.

FIG. 12 is a set of chemical structures of enterobactin.

FIG. 13 is a schematic diagram of the enterobactin gene cluster in E. coli.

FIG. 14 is a schematic diagram of the enterobactin synthetase.

FIG. 15 is a schematic diagram showing the priming of apo-EntB and apo-EntF into their active form by EntD.

FIG. 16 is a chemical reaction of the conversion of chorismate to DHB by EntC, EntB, and EntA.

FIG. 17 is a schematic diagram showing the formation of the DHB-Ser acyl enzyme intermediate by EntE, EntB, and EntF.

FIG. 18 is a schematic diagram showing the TE domain of EntF catalyzing elongation and macrocyclization, which releases enterobactin.

FIGS. 19-27 are schematic diagrams showing synthesis of enterobactin by enterobactin synthetase in a stepwise manner.

FIG. 28 is a schematic diagram showing export of enterobactin from a bacterial cell and importation of Fe³⁺-enterobactin into the cell.

FIG. 29 is a set of images showing selection for E. coli with Ent synthetase activity on iron-deficient media grown in the presence of an iron chelator, 2,2′-dypryridyl.

FIG. 30 is a schematic diagram showing the selection of EntF with a heterologous A domain.

FIG. 31 is a set of photographs showing decreased activity of chimeras in an in vivo assay of EntF activity.

FIG. 32 is a schematic diagram showing increased activity of EntF-A_{Het} selectants following one and two rounds of selection.

FIGS. 33A and 33B are images of colonies. FIG. 33A shows satellite colonies growing around a hit colony. FIG. 33B is a set of images showing growth of EntF-A_{Het} colonies under selective conditions.

FIG. 34 is a set of SDS-PAGE gels showing purification of WT and several EntF-A_{Het} selectants including D1165A, 410, 410-H4, 410B-02, and 410B-06.

FIG. 35 is a sequence alignment of the amino acid sequences of four EntF-A_{Het} selectants.

FIGS. 36-38 are structural models of EntF-A_{Het} selectants.

FIG. 39 is a schematic diagram showing the chemical structure of bacillomycin D and the gene cluster of the bacillomycin synthetase genes.

FIG. 40 is a schematic diagram showing the domain organization of the bacillomycin synthetase.

FIG. 41 is an image showing lipopeptide activity of bacillomycin on a fungal overlay.

FIG. 42 is a schematic diagram showing that swapping the A_{Ser} domain for an A_{Asn} domain in BmyC results in no production of bacillomycin from its synthetase.

FIG. 43 is a schematic diagram demonstrating that mutating the substituted A_{Asn} domain results in increased production from the bacillomycin synthetase, and that the substitution of the A_{Asn} for the A_{Ser} domain results in production of an altered product.

FIG. 44 is a schematic diagram showing the protein-protein interactions that take place between EntB and other components of the enterobactin synthetase including EntD, EntE, and EntF.

FIG. 45 outlines the shotgun alanine scanning technique utilized to assay protein-protein interactions in enterobactin synthetase.

FIG. 46 is an image and a table showing the changes produced by the shotgun alanine scanning technique.

FIG. 47 is a schematic diagraph and set of photographs showing the selection conditions utilized to identify variants that retain enterobactin syntehetase activity.

FIG. 48 is an image and a table showing analysis of surviving clones under the selective conditions of FIG. 47. Ratios of WT/Ala residues at the various positions and calculated changes in free energy are shown.

FIG. 49 is an image and set of tables showing analysis of mutation at alanine mutations to lysine or glutamate at positions 250 and 253.

FIG. 50 is an image and a table showing analysis of changes at position 249 from Met to Ala, Val, or Thr.

FIG. 51 is a schematic diagram showing the reduction in enterobactin production of M249A EntB as compared to WT EntB in vitro.

FIG. 52 is a schematic diagram showing the reduction in condensation of DHB with Ser-S-NAC in the M249A EntB as compared to WT EntB.

FIG. 53 is a set of graphs showing that Sfp-catalyzed Ppantylation and EntE-catalyzed salicylation are not affected by the M249A EntB as compared to WT EntB.

FIG. 54 is a schematic diagram showing recognition by Type II PKSs may be similar to recognition in enterobactin synthetase.

FIGS. 55 and 56 are images showing the EntB-ArCP (aryl carrier protein), with preferred regions for randomization highlighted.

FIG. 57 are images showing the use of antifungal (e.g., anti-yeast) screens as a an indicator for biological activities in eukaryotes. The example shown is a zone-of-inhibition study of the immunosuppressant rapamycin, from its cognate producer organism S. hygroscopus.

FIGS. 58A and 58B are schematic diagrams showing synthesis of enterobactin and the protein-protein interactions required for enterobactin synthesis. FIG. 58A shows the enterobactin synthetase, consisting of four proteins: EntBDEF. The following abbreviations are used for domain functions A, adenylation; ICL, isochorismate lyase; C, condensation; PCP, peptidyl carrier protein; TE, thioesterase. FIG. 58B shows protein-protein interactions required for enterobactin production. EntB-ArCP must contact (i) EntD (or other PPTases), (ii) EntE, and (iii) EntF at various points during the biosynthetic cycle.

FIGS. 59A and 59B are schematic diagrams showing the structure of EntB-ArCP. FIG. 59A shows ribbon representation of the EntB-ArCP structure. FIG. 59B shows surface of EntB-ArCP color coded for degree of conservation where red (D244, F264, A268) is high, orange (G242, M249) is intermediate, and green (light grey; EntF Face, and PPTase Face) is low. Serine 245 is shown in blue. The residues that comprise the differential PPTase and EntF interaction faces are indicated.

FIG. 60 is a schematic diagram showing structure-based design of the H1, L1A, and L1B libraries. The sequence of EntB-ArCP from CFT073 is shown with helices 1 through 4 (H1-H4) and loops 1 and 2 (L1 and L2) indicated. The residues subjected to shotgun alanine scanning randomization are shown in red (library H1), blue (library L1A), and green (library L1B). The residue A216 was allowed to vary among Ala (WT), Lys, Glu, and Thr. The phosphopanthetheinylated S245 is indicated with an asterisk.

FIGS. 61A and 61B are schematic diagrams showing mapping of conservation data from the H1, L1A, and L1B libraries onto the apo-EntB-ArCP crystal structure. FIG. 61A shows surface representation color-coded for degree of conservation where red (D244) is high, orange (G242) is intermediate, and green (light grey) is low. The phosphopantetheinylated S245 is shown in blue. FIG. 61B shows a ribbon diagram (same orientation) with the sidechains for the highly conserved residues shown.

FIGS. 62A and 62B are schematic diagrams showing that the sidechains of L238 and L243 point toward the ArCP structure core (FIG. 62A; surface of the ArCP is represented as mesh) and the putative charge-charge interactions between D234 of loop 1, and R219 and K215 of helix 1 (FIG. 62B).

FIGS. 63A and 63B graphs showing the time course of phosphopantetheinylation of EntB-ArCP (WT or G242A mutant) by EntD and Sfp.

FIGS. 64A-64C are HPLC traces showing phosphopantetheinylation of WT (FIG. 64A), D244A (FIG. 64B), and D244R (FIG. 64C) by EntD as monitored by HPLC (reaction times 20 mins and 2 hrs). The peaks corresponding to apo and holo are indicated.

FIG. 65A is a schematic diagram showing EntD is a PPTase that primes EntB and EntF. EntE loads DHB onto holo-EntB-ArCP. EntF is a four-domain NRPS elongation/termination module (C-A-T-TE). The A domain of EntF catalyzes loading of the T domain with Serine. The C domain then mediates condensation of Serine with DHB loaded on EntB ArCP. The TE domain elongates DHB-Ser and macrocyclizes the DHB-Ser trimer to form enterobactin.

FIG. 65B is a schematic diagram showing the cascade of enterobactin biosynthesis reactions that involve the EntF T domain. The interdomain interactions that must occur for each step are shown.

FIGS. 66A and 66B are schematic diagrams showing a homology model of the EntF T Domain. FIG. 66A shows a sequence alignment of the EntF T domain with TycC3-PCP that was used for generation of the EntF T domain homology model. FIG. 66B shows a ribbon diagram of the homology model of EntT T domain. Residues of EntF T domain that were subjected to shotgun alanine scanning are indicated.

FIGS. 67A and 67B are schematic diagrams showing conserved residues from combinatorial mutagenesis and selection. Ball and stick (FIG. 67A) and surface (FIG. 67B) representation of residues on the EntF T domain model that were highly conserved in the iron-deficient selection. In the surface representation, the conserved residues are shown in red (L1007, M1030, and G1027), the nonconserved residues are shown in green (light gray), the phosphopantetheinlyated Ser (1006) is shown in yellow, and unscanned residues are shown in darker gray.

FIGS. 68A and 68B are graphs showing initial characterization of EntF T domain mutants. FIG. 68A shows phosphopantetheinylation of EntF T domain (wt and mutants) by EntD. Time courses of radiolabeled holo-EntF production are shown. FIG. 68B shows time courses for enterobactin production for EntF (wt and mutants).

FIGS. 69A and 69B are schematic diagrams and graphs showing characterization of A-T and C-T domain-domain interactions for the EntF T domain mutants G1027A and M1030A. FIG. 69A shows a time course for [¹⁴C]-Ser incorporation onto EntF (wt and mutants). FIG. 69B shows HPLC analysis of DHB-Ser condensation products on tridomain C-A-T constructs. The EntF tridomains (wt and mutants) were preloaded with Ser and DHB were added to start the condensation reaction in the presence of EntEB. Protein bound Ser or DHB-Ser were released by KOH treatment.

FIG. 70 is a graph showing acyl-transfer assay for T-TE interaction. The PPTase Sfp was used to load 1-[¹⁴C]-acetyl-pantetheine onto the T domain (loading phase). In wild-type EntF, this acyl group is transferred to the downstream TE domain and 1-[¹⁴C]-acetyl-O-TE is hydrolyzed to liberate [¹⁴C]-acetate (release phase). When T-TE interaction is impaired, the covalent label remains stably incorporated.

FIGS. 71A-71C are schematic diagrams showing a model for interaction between a T domain and its Downstream partner. FIG. 71A shows the surface (blue) and ribbon (green) representation of EntB T domain (structure prepared from PDB code: 2FQ1) in trans interaction with EntF C domain. The helix III residues (F264 and A268) which are responsible for interaction with EntF C domain are shown in red (labeled “helix III”). The helix II residue M249 is shown in orange. FIG. 71B shows EntF T domain (homology model) in cis interaction with EntF TE domain. The helix III residues (G1027 and M1030), which are responsible for T-TE interaction, are shown in red (labeled “Helix III”). FIG. 71C shows a model of helix III as communication motif for T-downstream domain interaction.

### DETAILED DESCRIPTION

The present invention provides methods for generating a modified assembly line. These modified assembly lines are useful for producing novel compounds (e.g., NRPs and PKs) that have activities including but not limited to antimalarial, immunosupressory, antitumor, anticholestrolemic, antibiotic (e.g., antibacterial), and antifungal activities.

### Assembly Lines

Assembly lines multimodular enzymes composed of individual domains arranged on one polypeptide or on a plurality of interacting polypeptides. Each individually folded “globule” is called a domain. Domains are organized into fundamental units called modules, which are defined operationally, as a set of domains responsible for incorporating one monomer into the growing chain. The complete set of modules responsible for assembling a natural product or the precursor of a natural product is called an assembly line (e.g., a synthase or synthetase).

The product of an assembly line may be a precursor to a product that undergoes further modification by, e.g., glycosyltransferases (e.g., the glyclosyltransferases that add sugars to the erythromycin aglycone) or oxidases (e.g., oxidases that form aryl-ether and aryl-aryl crosslink in the vancomycin-family glycopeptides). The modifications may be necessary for the natural product to be active. If the product of the assembly line itself does not have biological activity, then either (i) the new product may still be recognized by the enzyme catalyzing the further modification (e.g., oxidases and glycosyltransferases) that recognized the product of the original assembly line, or (ii) the new product may be a precursor for a semisynthetic drug. The precursor may then be modified by standard organic synthesis techniques, thereby transforming the precursor into an active drug. Taxol, for example, is produced in this manner.

### Nonribosomal Peptide Synthetases (NRPS)

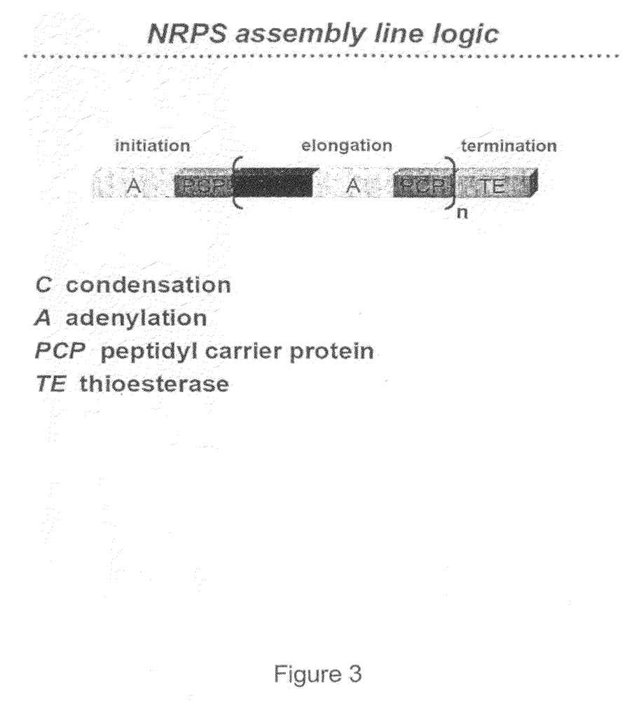

The following domains may be included within an NRPSs: C (condensation), Cy (heterocyclization), A (adenylation), T (thiolation) or PCP (peptidyl carrier protein), TE (thioesterase), E (epimerization), MT (methyltransferase), Ox (oxidase), and Re (reductase) domains. Nonribosomal peptide synthetases generally have the following structure: A-T-(C-A-T)ₙ-TE where A-T is the initiation module, C-A-T are the elongation modules, and TE is the termination module (see FIG. 3). Within the individual modules, the following variations may, for example, occur: C is replaced by Cy, E, MT, Ox, or Re are inserted; TE is replaced by C or Re. A complete assembly line may have an initiation module, a termination module, and somewhere between zero and n-2 elongation modules, where n is the number of monomers in the polymeric product. Exceptions to this rule may exist; e.g., the enterobactin synthetase, in which the TE domain acts as an oligomerase, so although it only has two modules, it hooks three of these dimeric products together to form a hexameric product.

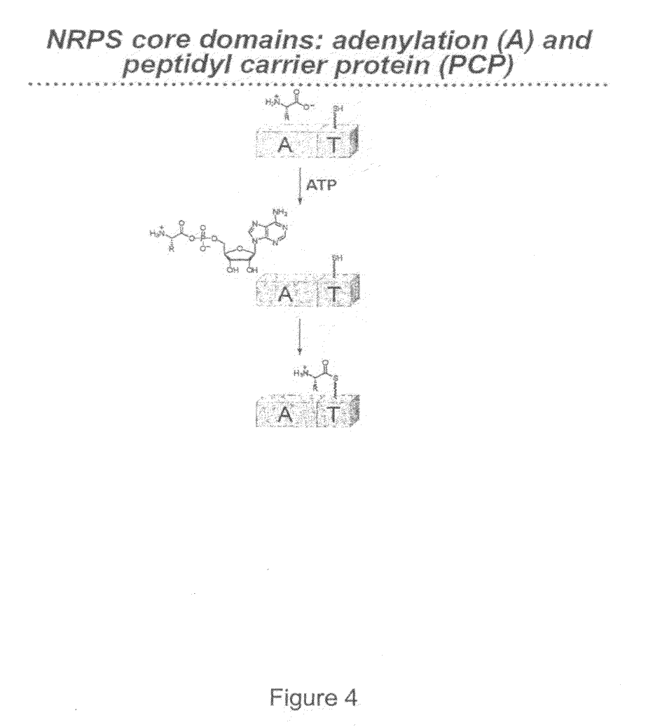

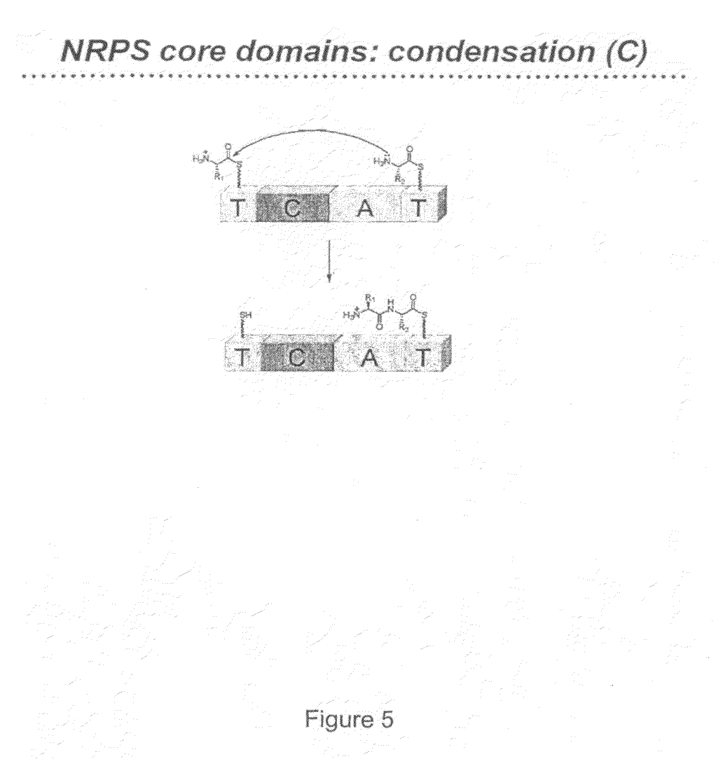

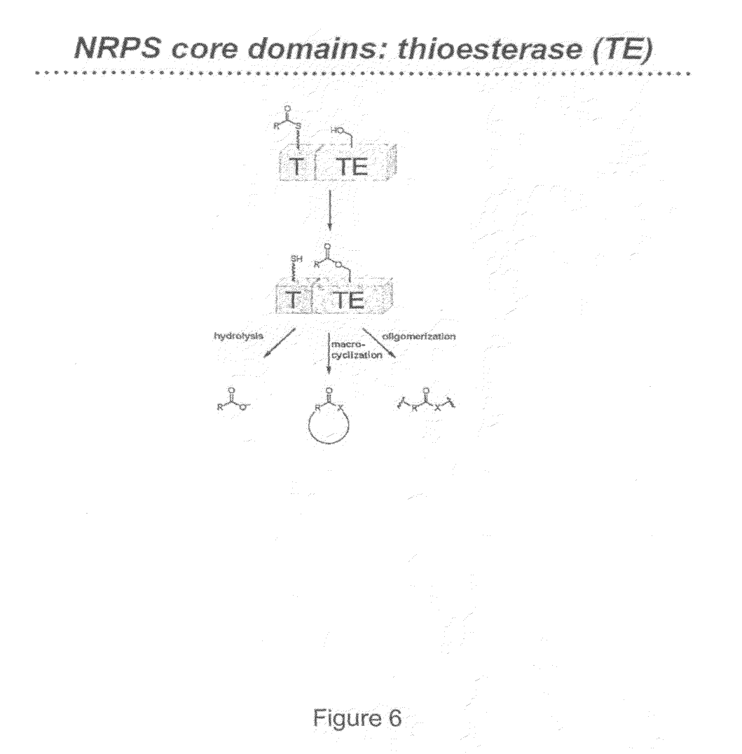

The NRPS core domains include the A and PCP (or T) domains (FIG. 4). This figure shows how a monomer is attached (using ATP) to the T domain of a module. In the elongation step, the monomer is transfered from the T domain of one module to the T domain of the next module. This transfer involves the C domain of the elongation module (FIG. 5). The final step of NRP synthesis is performed by the TE domain, which catalyzes a hydrolysis, a macro-cyclization, or oligomerization reaction (FIG. 6).

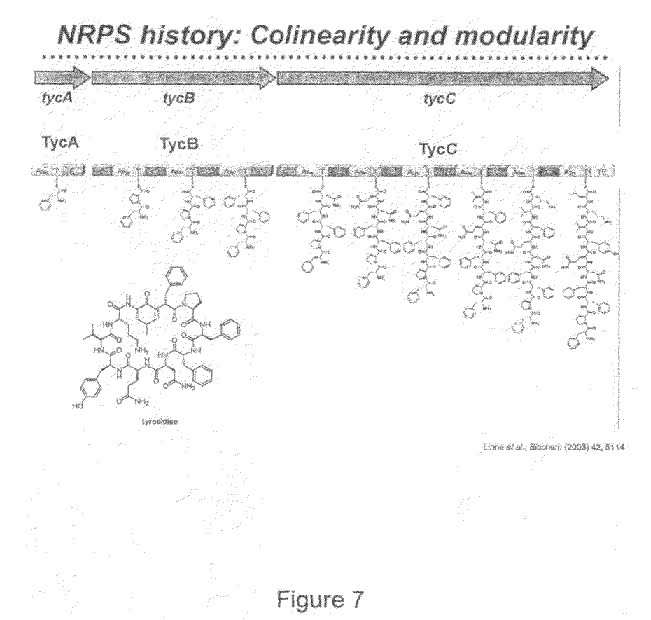

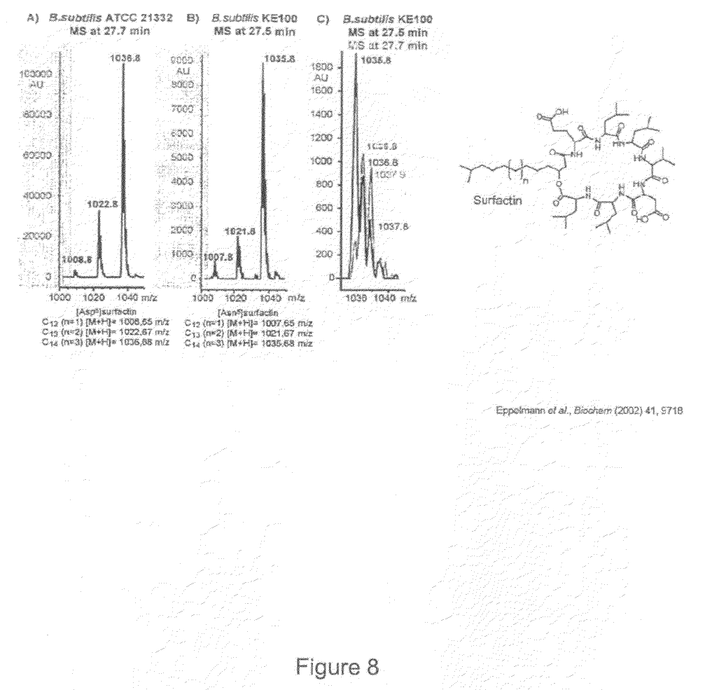

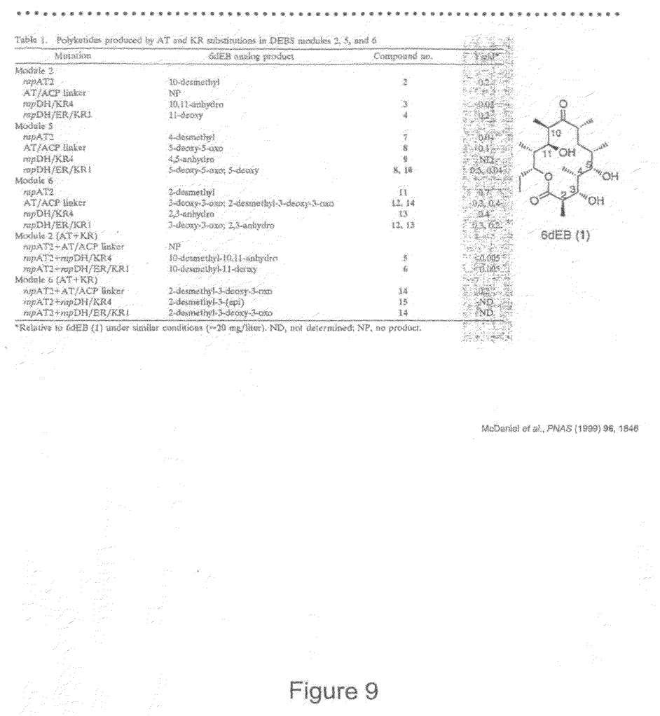

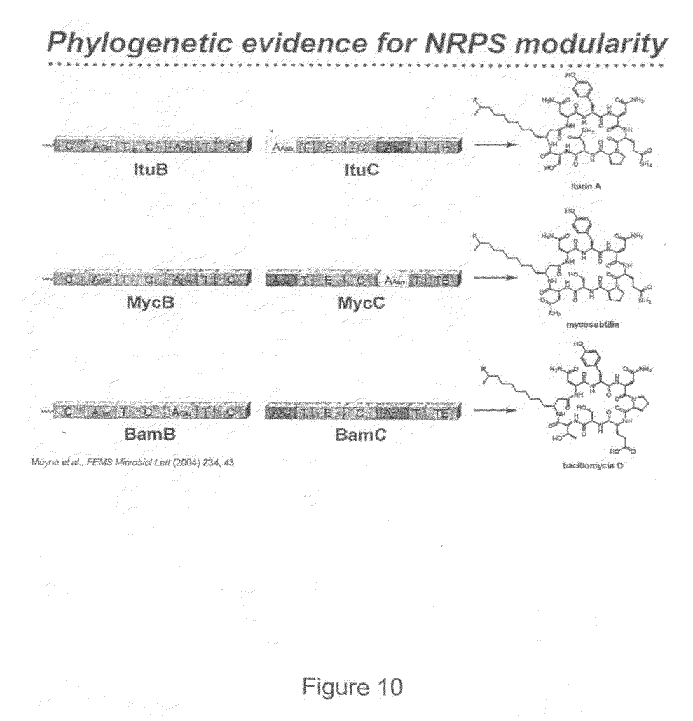

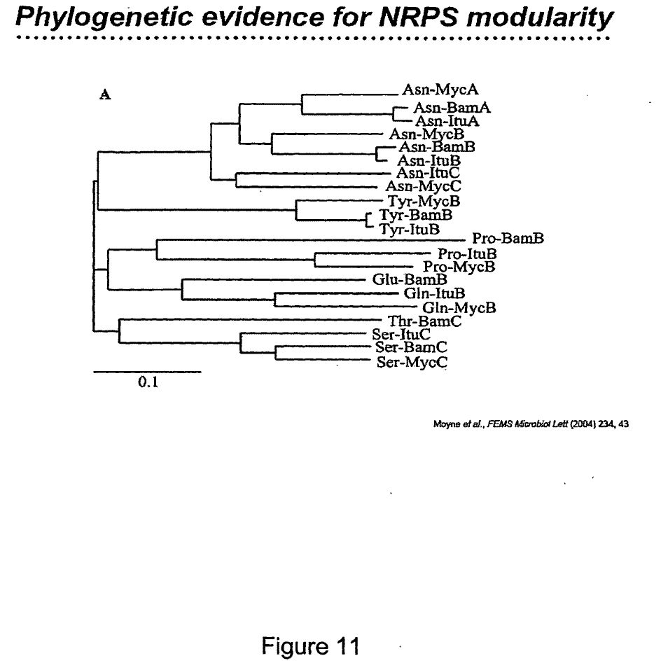

NRPSs are generally modular, and the series of catalytic steps moves from the amino to carboxy terminus of each polypeptide that makes up the NRPS. For example the NRPS that produces typrocidine is made of three genes producing three polypeptides. TycA contains the initiation module; TycB contains three elongation modules, and TycC contains six additional elongation modules plus a termination module (FIG. 7; Linne et al., Biochemistry (2003) 42, 5114).

### Polyketide Synthetase

The following domains may be included within a PKS: KS (ketosynthase), AT (acyltransferase), T (thiolation), KR (ketoreductase), DH (dehydratase), ER (enoylreductase), TE (thioesterase). PKSs generally have the following structure: AT-T-(KS-AT-T)n-TE. AT-T is the initiation module, KS-AT-T are the elongation modules, and TE is the termination module. The structure of a PKS is very similar to NRPS structure. There are many examples (e.g., yersiniabactin, epothilone, bleomycin) of hybrid PKS-NRPS systems in which both types of assembly line are pieced together to form a coherent unit. Within each PKS module, one either finds a KR, a KR and DH, a KR and DH and ER, or no additional domains. These extra domains within a module determine the chemical Functionality at the beta carbon (e.g., carbonyl, hydroxyl, olefin, or saturated carbon).

### Products of Assembly Lines

Assembly lines produce, for example, NRPs, PKs, and combinations/hybrids of NRPs and PKs. A comparison between NRPs and ribosomal peptides is shown in Table 1. In one example of a NRPS, epothilone synthetase has a molecular mass of ˜1800 kDa and includes six polypeptides, whereas the ribosome is ˜2600 kDa and includes 55 proteins and 3 rRNAs.

TABLE 1

| | Nonribosomal peptides | Ribosomal peptides |
|---|---|---|
| Producer organisms | soil bacteria | all walks of life |
| | filamentous fungi | |
| Monomers | >300 amino, hydroxy, | 20 proteinogenic amino |
| | and aryl acids | acids + Se-Cys and |
| | | Py-Lys |
| Topological | linear | linear (except in the |
| Conformation | cyclic | case of S—S bonding) |
| | branched cyclic | |
| Biosynthetic | one synthetase | one ribosome |
| machinery | one product | many products |

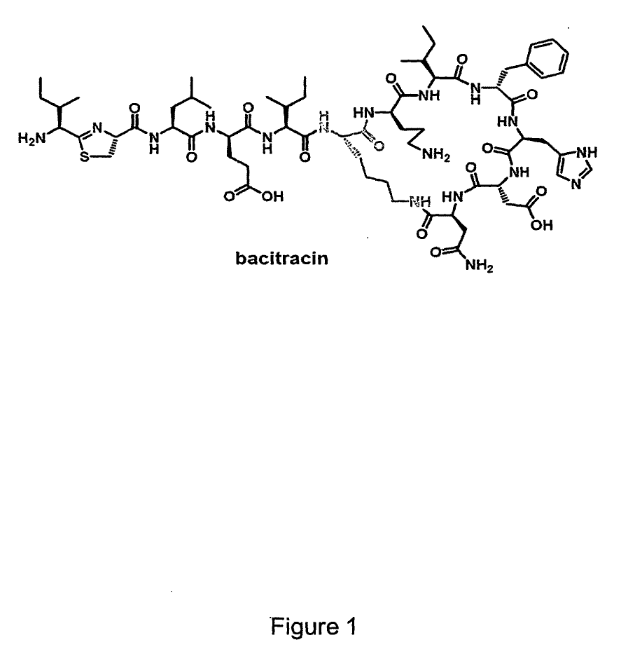

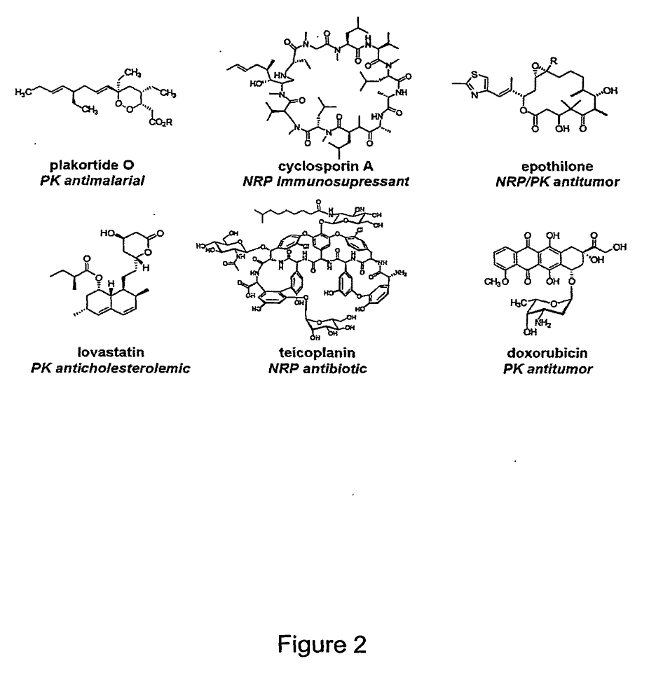

As noted in Table 1 and shown in FIG. 1, NRPs contain chemical features not found in ribosomal peptides. Examples of NRPs, PKs and NRP/PK hybrid include plakortide O, cyclosporin A, epothilone, lovastin, teicoplanin, and doxorubicin (FIG. 2).

### Identification of Modified Assembly Lines

The present invention includes identification of modified assembly lines (MAL) using a screen or selection. Any screen or selection method standard in the art may be used to create the MALs of the present invention. Typically, one or more random mutations is introduced into a domain to create a variant domain of a nucleic acid encoding a NPP, PK or PK-NRP hybrid. Selective pressure or a screen is then applied to cells encoding the assembly lines having the variant domains. The steps of mutation and selection may be repeated. Suprisingly, despite many prior unsuccessful attempts to alter assembly lines and then natural products, we have discovered that the approach of directed evolution of specific domains rapidly and readily produces MALs having improved biosynthetic capacity and/or synthesizing novel variants of natural products. Our findings have resulted in a method of enhancing production of natural products and creating new natural products even when little tertiary or quaternary structural information is available regarding the assembly line and the natural product is one which is inaccessible for chemical modification. We believe this approach has tremendous ramifications for the production of therapeutically important molecules.

### Screens

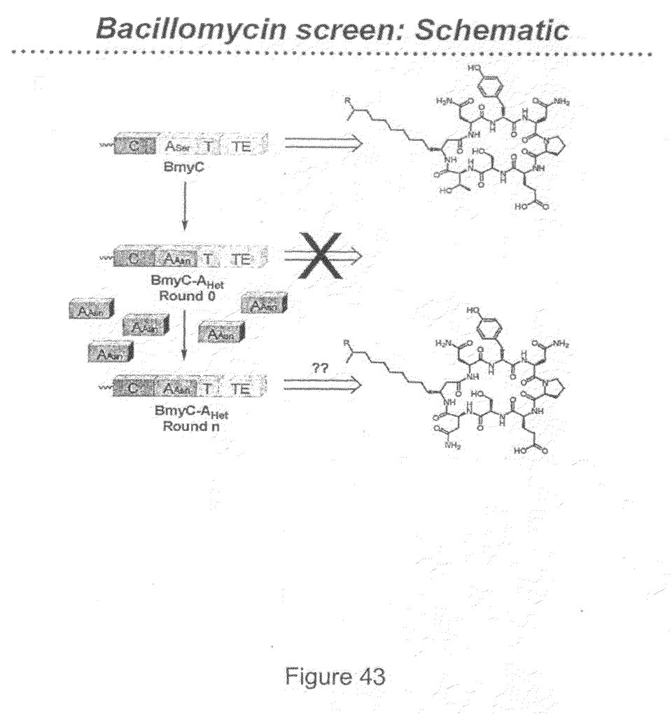

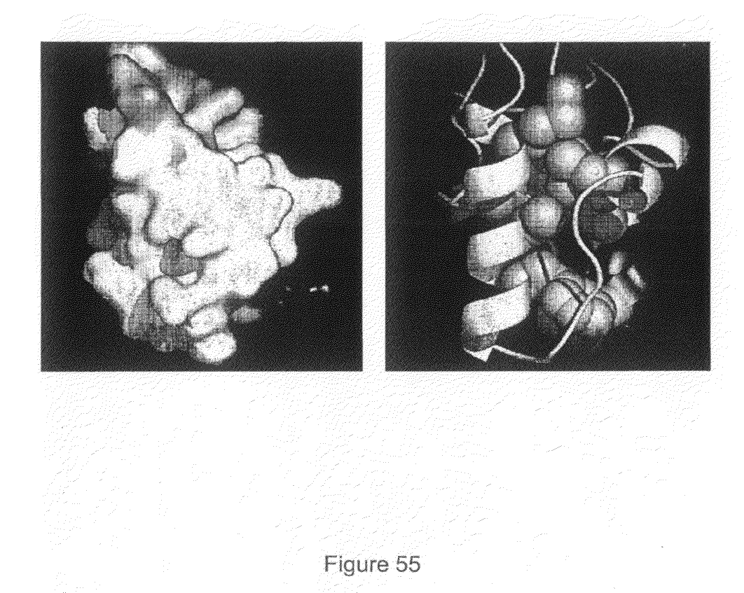

A preferred screen for secretion of a PK, NRP, or PK-NRP product includes a library of producer cells (produced by transformation with a library of plasmids or other vectors encoding a portion of the assembly line which are autologous or integrated) plated on top of a lawn of a tester strain. The tester strain may be bacterial or fungal strain sensitive to the product of the assembly line or predicted to be sensitive to the novel product produced by a modified assembly line (see FIGS. 43 and 55). When individual clones of the producer cells produce sufficient activity or amount of the product to which the tester strain is sensitive, the inhibition (or other modification in growth) of the tester strain is visible on the plate.

The readout for the screen may include, for example, fusing a metabolite-responsive promoter element to a reporter gene (e.g., luciferase or GFP) and screening by FACS. In this format, the metabolite-responsive promoter might be the target of a two-component system that normally senses the presence of the assembly line product and initiates a host self-protection response in the producing organism. For example, the Tet (tetracycline) repressor and the associated Tet-on and Tet-off plasmid constructs, which are standard in the art, could be used to perform such a screen.

### Selection

A selection maybe performed by growing two strains—a producer (e.g., a library of producers) and a tester—in culture (e.g., liquid or solid culture) together, and strains that successful produce the desired assembly line product win the competition with their unsuccessful counterparts and take over the population. An example of this assay is described by Arndt et al. ((1999), Microbiol 145, 1989-2000). Alternatively, selection for a trait in a single cultured strain is also possible using selective media conditions. Selection conditions for the biosynthetic products of assembly lines are known in the art.

### Generation of Libraries of Assembly Lines

Libraries of assembly lines may be generated using molecular biology methods standard in the art. Random mutagenesis of a domain or domains of an assembly line may be performed using known methods such as error prone PCR described herein. While we have discovered that functional MALs may be generated with as few as four mutations in a domain using our selection and screening protocols, it will be appreciated that the degree of variation introduced into a domain may be controlled by the practitioner.

### Mutating Domains

Mutagenesis may be accomplished by variety of means, including the GeneMorph® II EZClone Domain Mutagenesis Kit (Stratagene, La Jolla, Calif.). Error prone PCR is a method standard in the art and described in Beaudry and Joyce (Science 257:635 (1992)) and Bartel and Szostak (Science 261:1411 (1993)). This technique may be used to introduce random mutations into genes coding for proteins. Kits for performing random mutagenesis by PCR are commercially available, for example, the Diversify™ PCR Random Mutagenesis Kit (BD Biosciences, Mountain View, Calif.). Chemical mutation, radiation, and any other technique known in the art for modifying the nucleic acid sequence are appropriate for use in the present invention.

### EXAMPLES

The following examples are meant to illustrate the invention and should not be construed as limiting. Other examples of modified assembly lines can be found, for example, in Lai et al., Proc. Natl. Acad. Sci. USA 103:5314-5319, 2006, hereby incorporated by reference.

### Example 1

### Creation of a MAL for Enterobactin Synthesis

### The Enterobactin Assembly Line

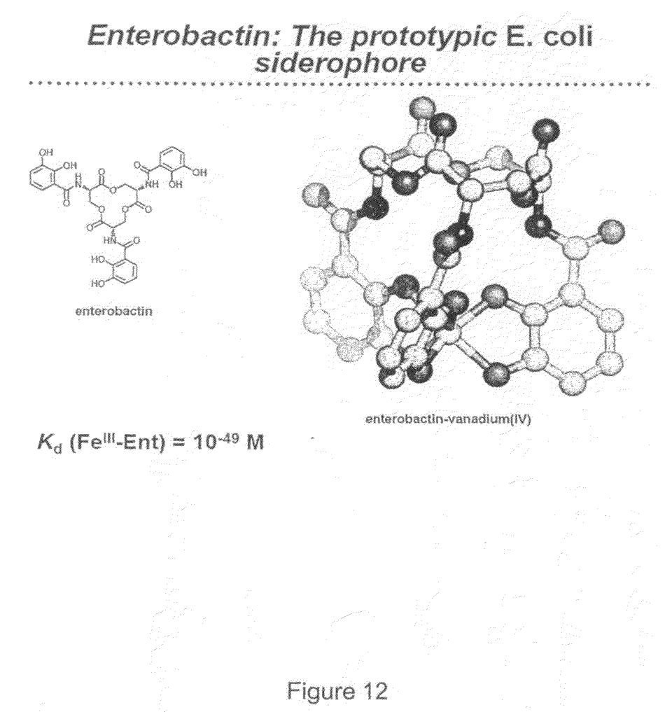

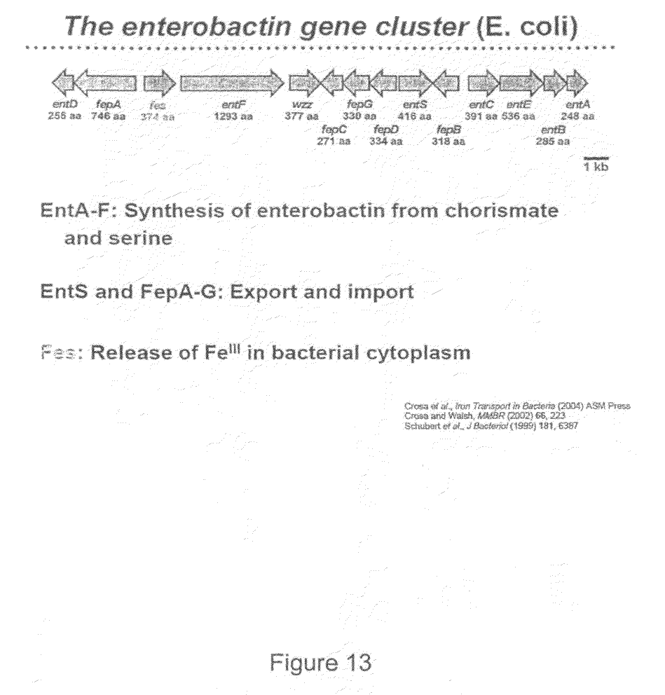

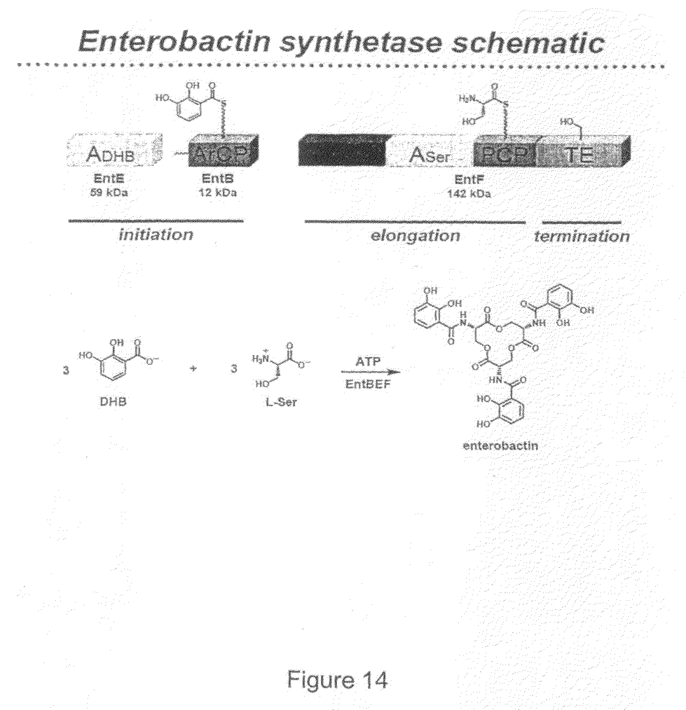

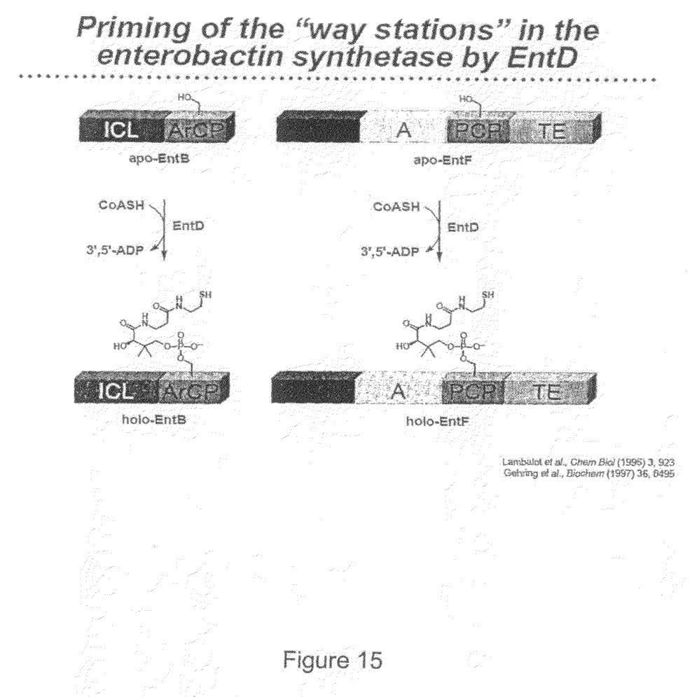

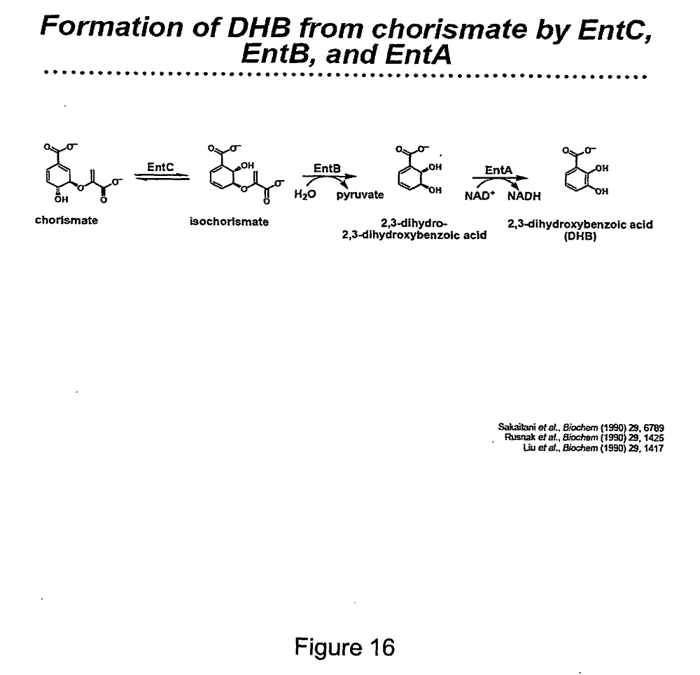

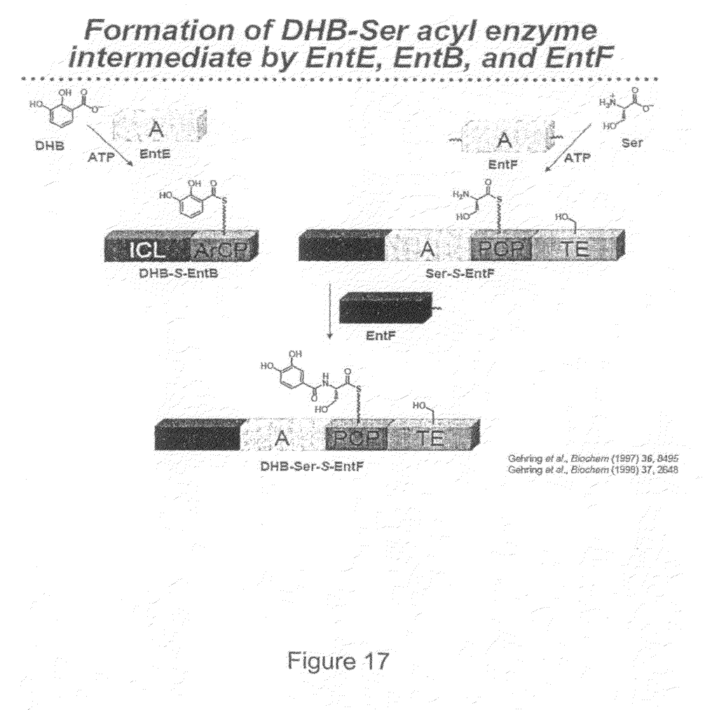

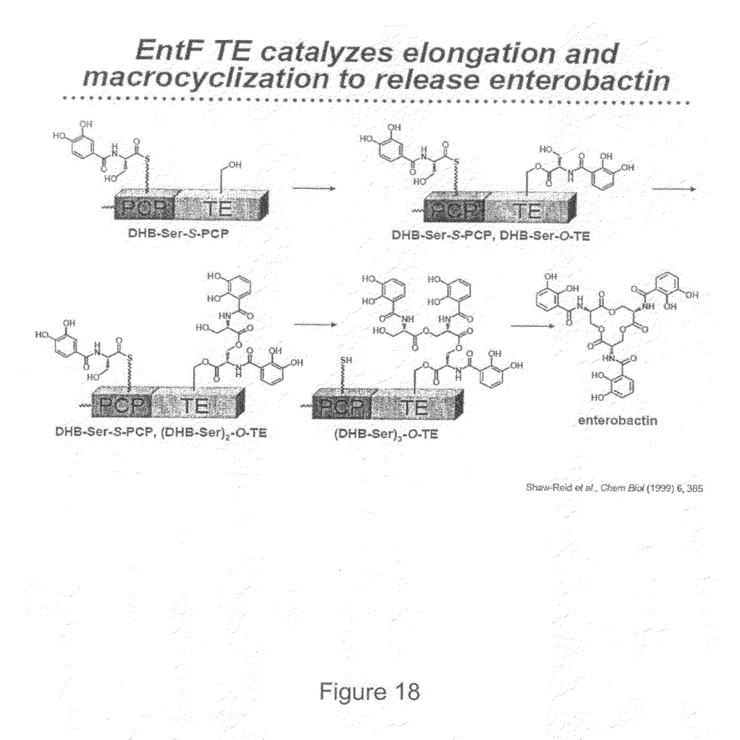

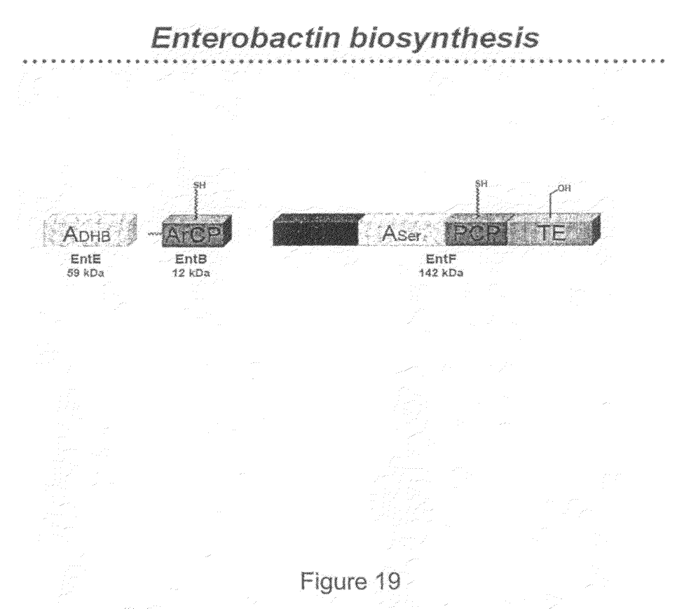

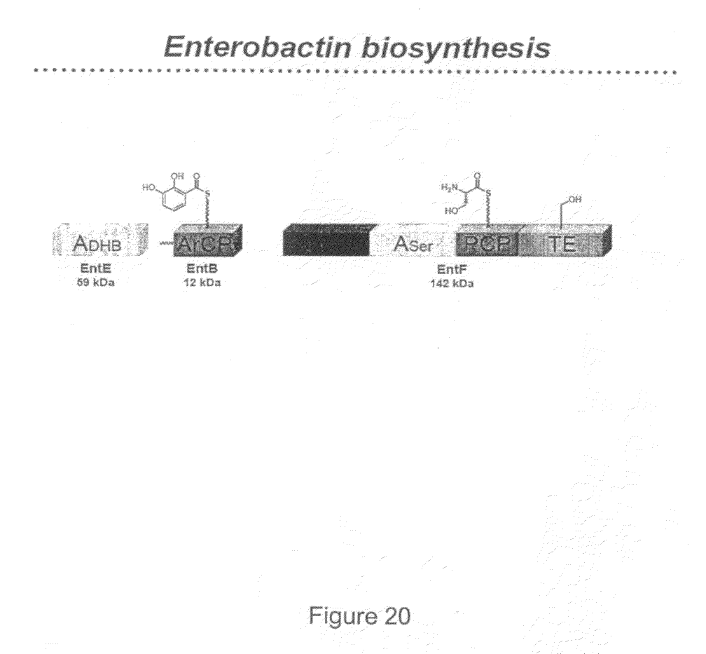

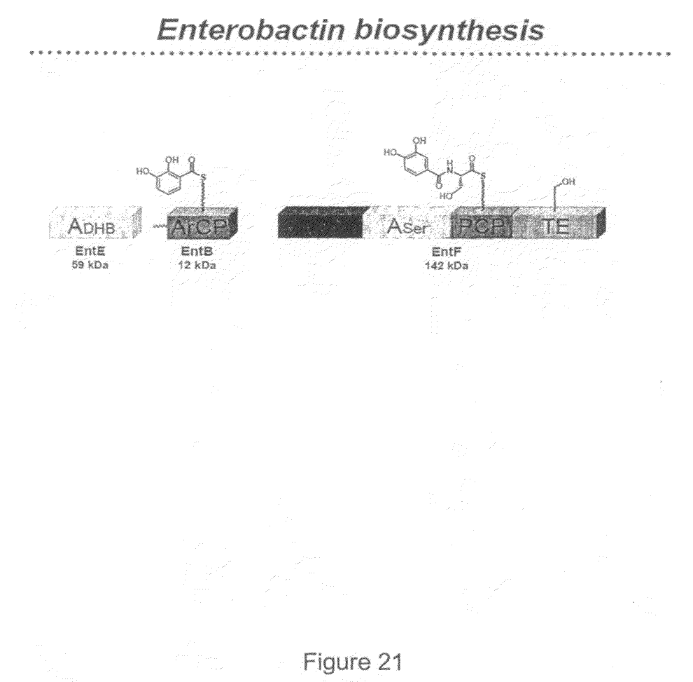

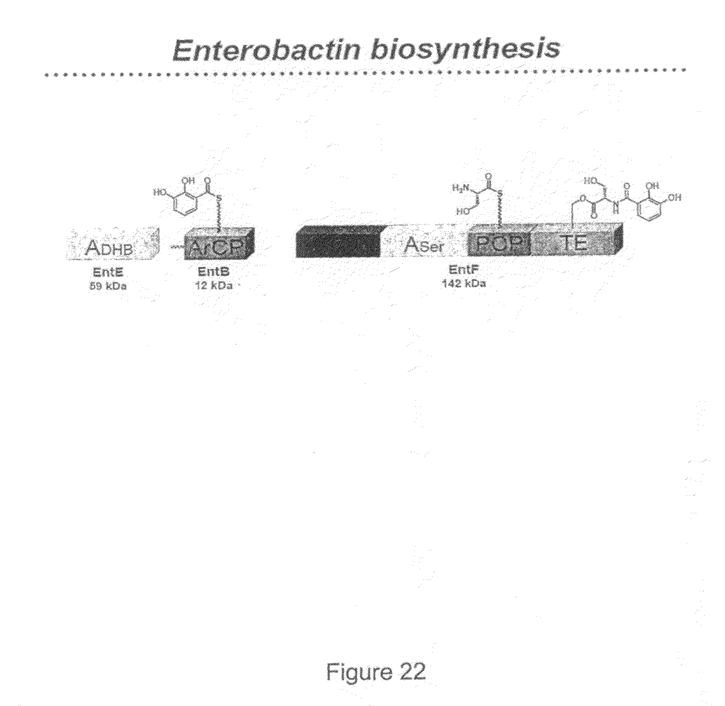

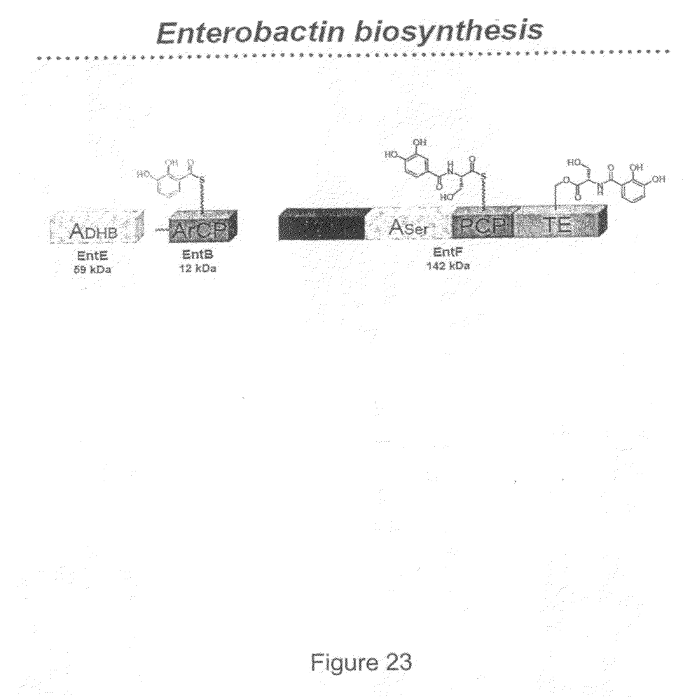

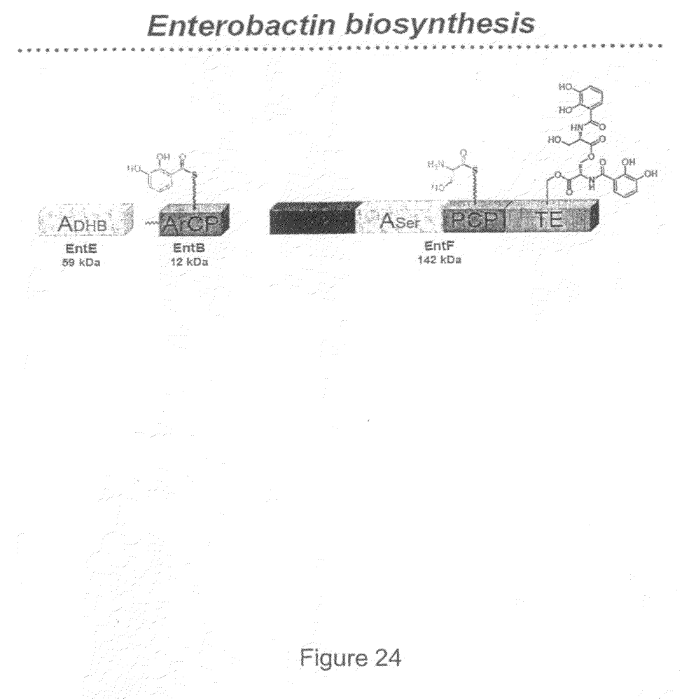

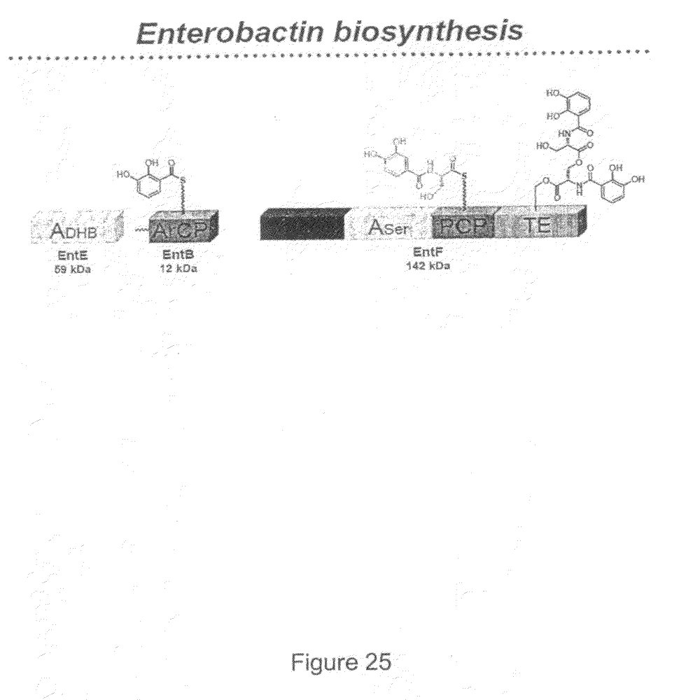

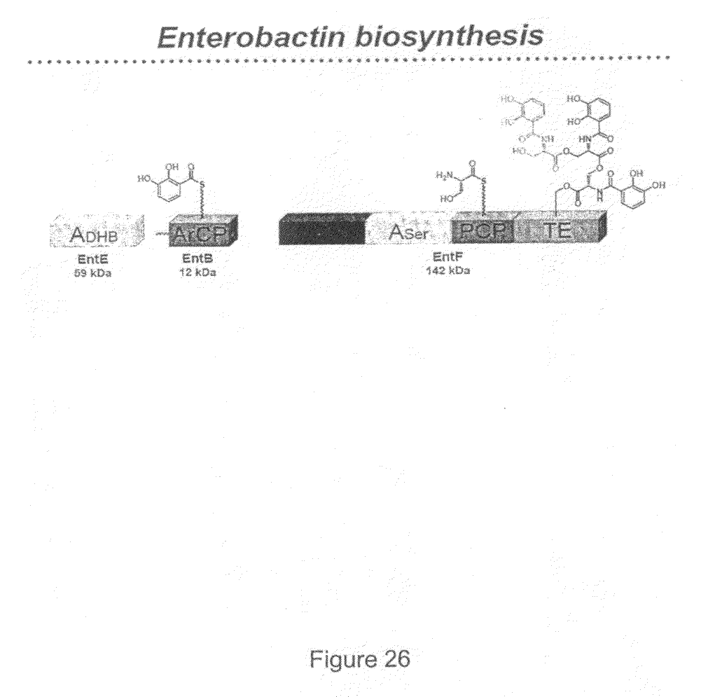

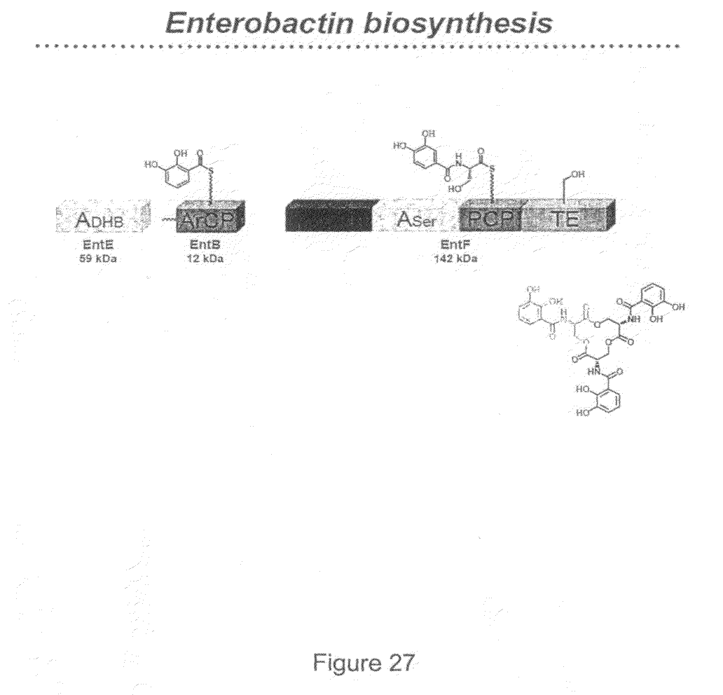

Enterobactin is a small, iron-chelating molecule known as a siderophore (FIG. 12). Siderophores are produced by bacteria (e.g., E. coli) and fungi. In E. coli, enterobactin is synthesized by enterobactin synthetase assembly line, components of which are produced by a single gene cluster (FIG. 13; Crosa et al., Iron Transport in Bacteria (2004) ASM Press; Crosa and Walsh, Microbiol Mol Biol Rev (2002) 66, 223; Schubert et al., J Bacteriol (1999) 181, 6387). EntA-EntF catalyze the synthesis of enterobactin from chorismate and serine; EntS and FepA-G are involved in import and export functions; Fes is involved in the release of Fe³⁺ into the bacterial cytoplasm. The assembly line that produces enterobactin comprises an initiation module, a single elongation module, and a termination module (FIG. 14). The initiation module comprises two domains, each on a separate polypeptide: EntE (the A domain for 2,3-dihydroxybenzoic acid; DHB) and EntB (the T domain). The elongation and termination modules comprise four domains (C, A, T, and TE), all part of EntF. EntB and EntF are produced in an inactive apo form, which is converted to its active form by EntD (FIG. 15; Lambalot et al., Chem Biol (1996) 3, 923; Gehring et al., Biochemistry (1997) 36, 8495). DHB is formed from chorismate by EntC, EntB, and EntA (FIG. 16; Sakaitani et al., Biochemistry (1990) 29:6789; Rusnak et al., Biochemistry (1990) 29, 1425; Liu et al., Biochemistry (1990) 29, 1417). DHB-Ser acyl enzyme intermediate is formed by EntE, EntB, and EntF (FIG. 17; Gehring et al., Biochemistry (1997) 36, 8495; Gehring et al., Biochemistry (1998) 37, 2648). The TE domain of EntF, in addition to catalyzing the release of the final product of the assembly line, also catalyzes the elongation and macrocyclization of enterobactin (FIG. 18; Shaw-Reid et al., Chem Biol (1999) 6, 385). Stepwise enterobactin biosynthesis by the assembly line is shown in FIGS. 19-27.

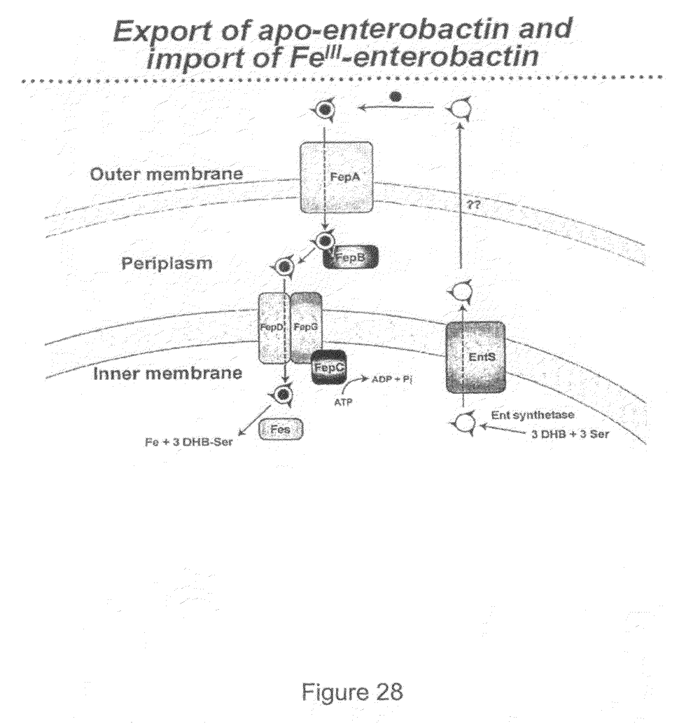

Following production of enterobactin, apo-enterobactin is exported from the E. coli cytoplasm by EntS. Enterobactin then interacts with Fe³⁺ and forms a complex, which is then imported across the outer membrane into the periplasm by FepA and transported by FepB to FepD and FepG, which import Fe³⁺-enterobactin into the cytoplasm, a reaction that is catalyzed by ATP hydrolysis of FepC. Fes converts the complex into Fe and DHB-Ser (FIG. 28).

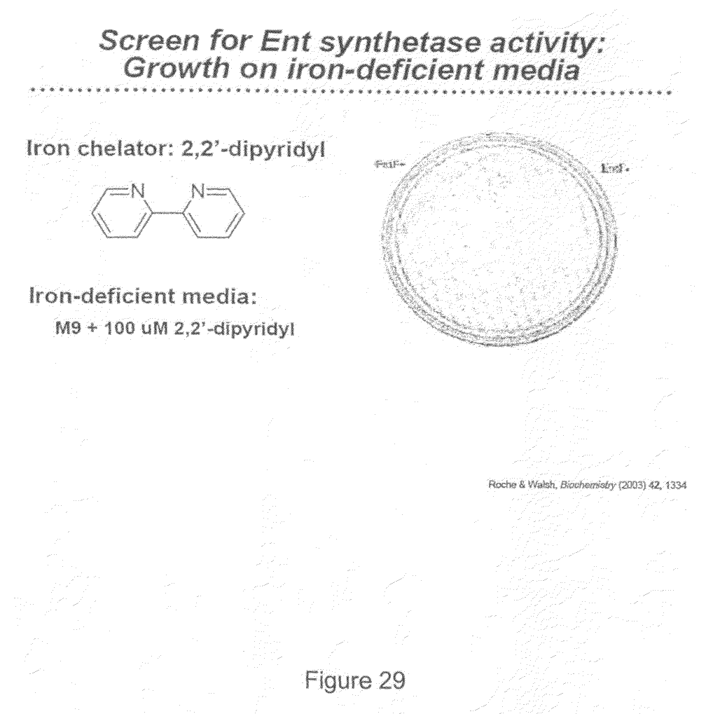

An EntF⁻ strain grown on minimal media in the presence of an iron chelator such as 2,2′-dipyridyl, is not capable of rapid growth, while an EntF⁺ does grow quickly (FIG. 29). When the EntF⁻ strain is complemented by a plasmid containing the EntF gene, it regains the ability to grow on iron-depleted media.

### Modification and Screening of the Enterobactin Assembly Line

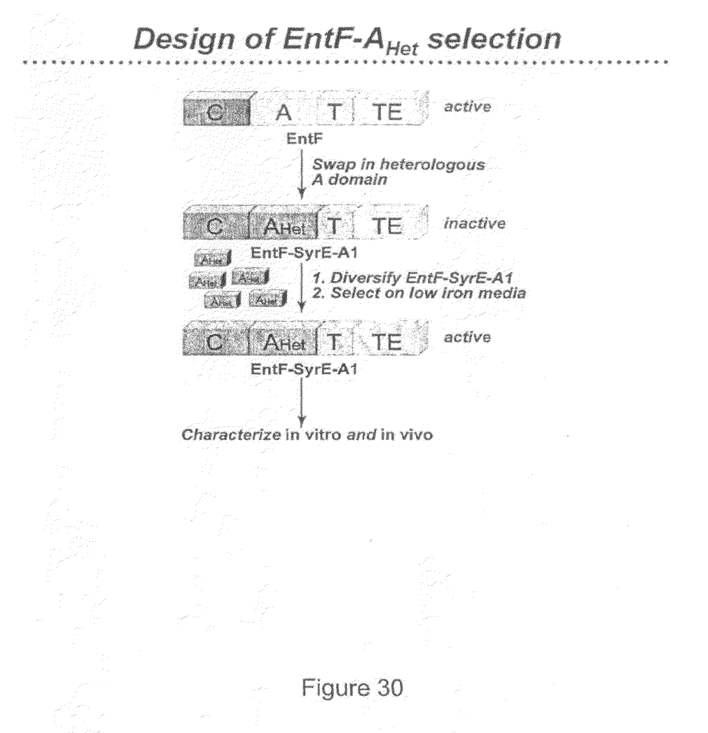

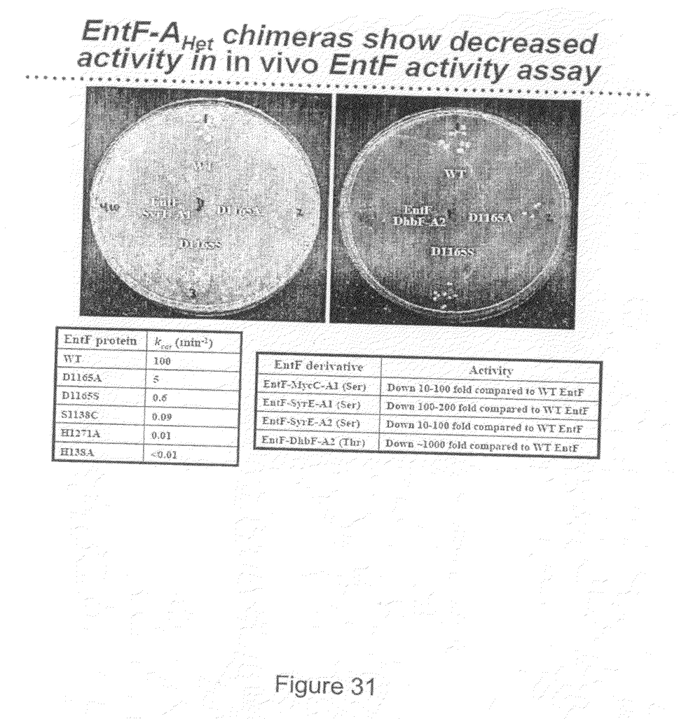

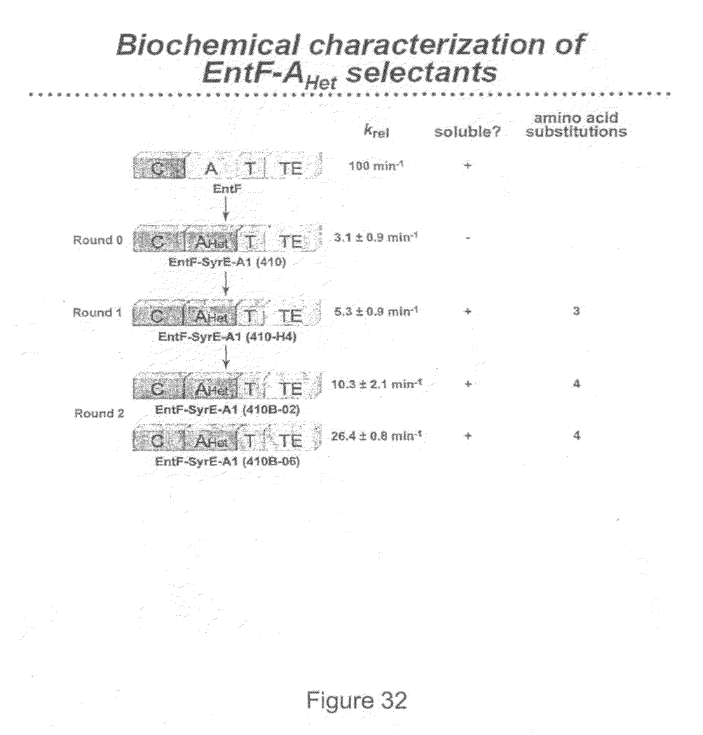

Using standard molecular biology techniques, the Ser-specific A domain from EntF was replaced with a Ser-specific A domain from the syringomycin synthetase (Pseudomonas syringae), SyrE-A1, creating a hybrid module, EntF-SyrE-A1 (FIG. 30). Despite the fact that SyrEA1 and EntF-A are catalytically equivalent, substitution of EntF-A with SyrE-A1 results in a 30-fold or greater reduction in activity as measured biochemically (FIGS. 31 and 32), and cells harboring EntF-SyrE-A1 exhibit a substantially reduced growth rate on iron-depleted media. Libraries of EntF-SyrEA1 assembly lines having variant domains were prepared by introducing mutations into the heterologous A domain using mutagenic (error prone) PCR, and these variants were transformed into the entF:cat strain and plated on iron-deficient media. The largest colonies, which should correspond to the clones harboring EntF-SyrE-A1 derivatives with increased activity, were picked and evaluated in a secondary screen of enterobactin production, which assays growth on iron-deficient media at lower cell density. The most active of these clones (410-H4) was chosen for a second round of diversification and selection.

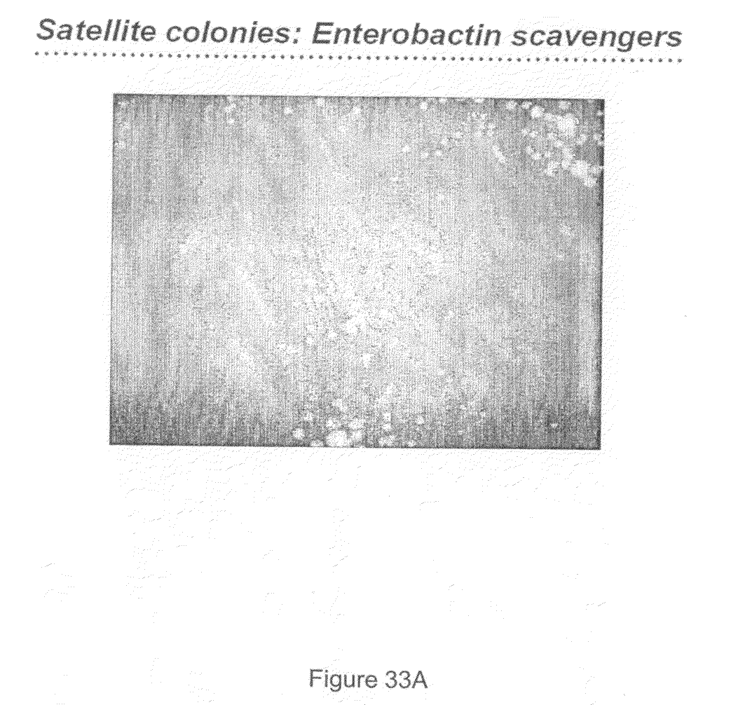

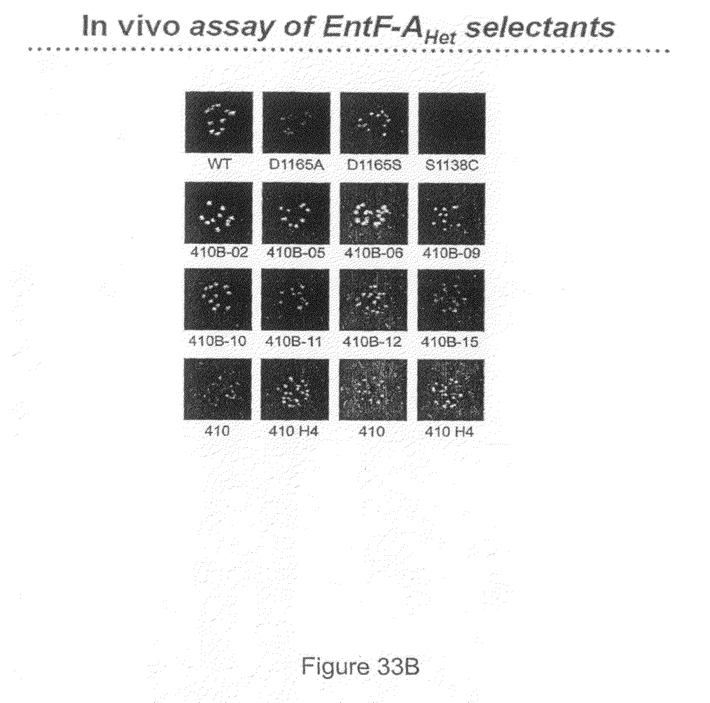

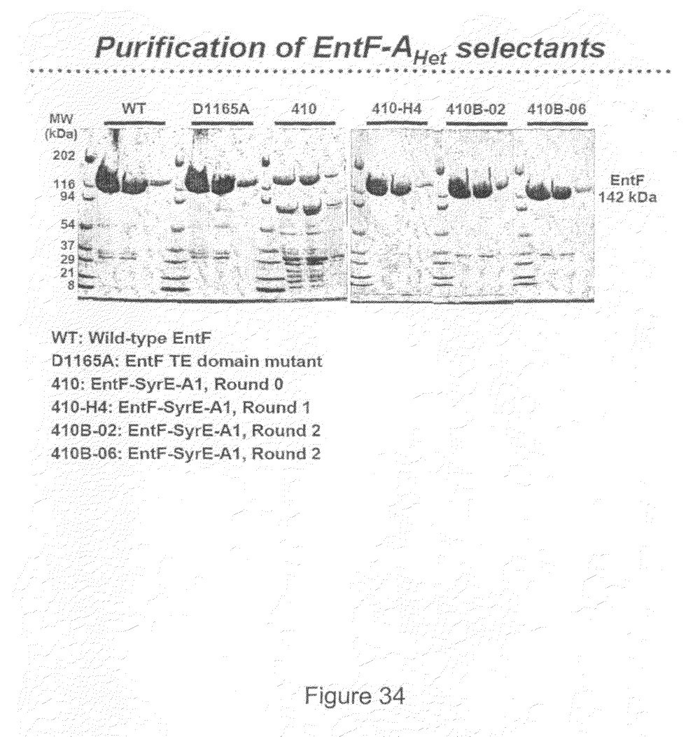

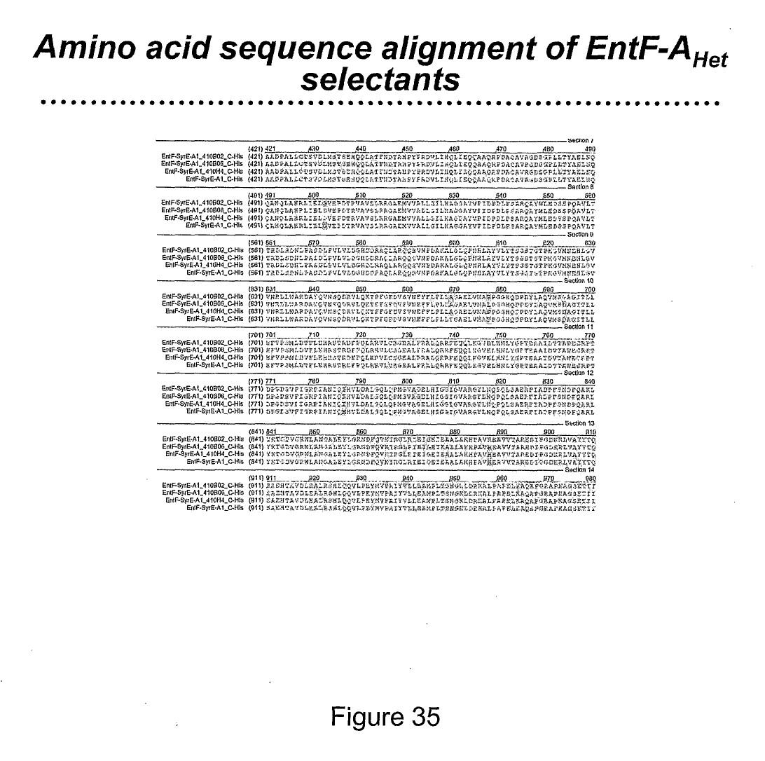

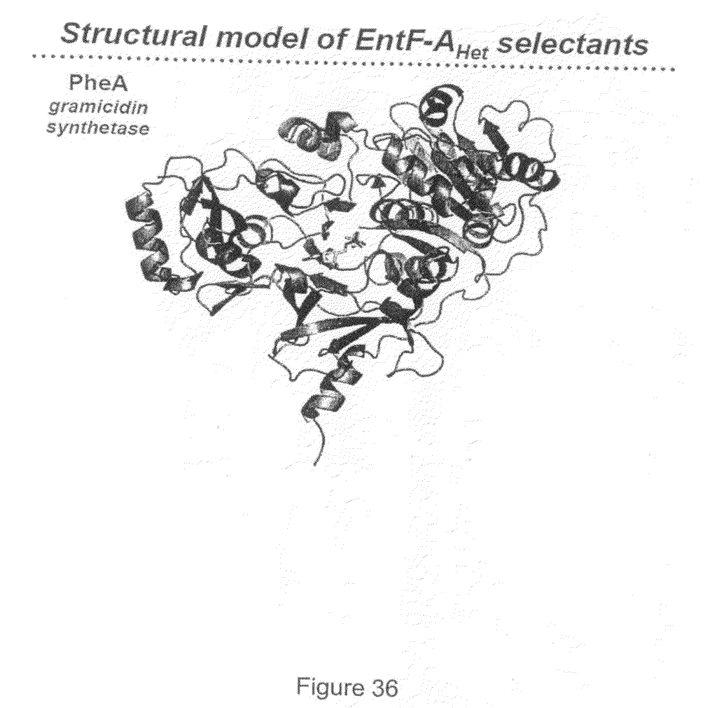

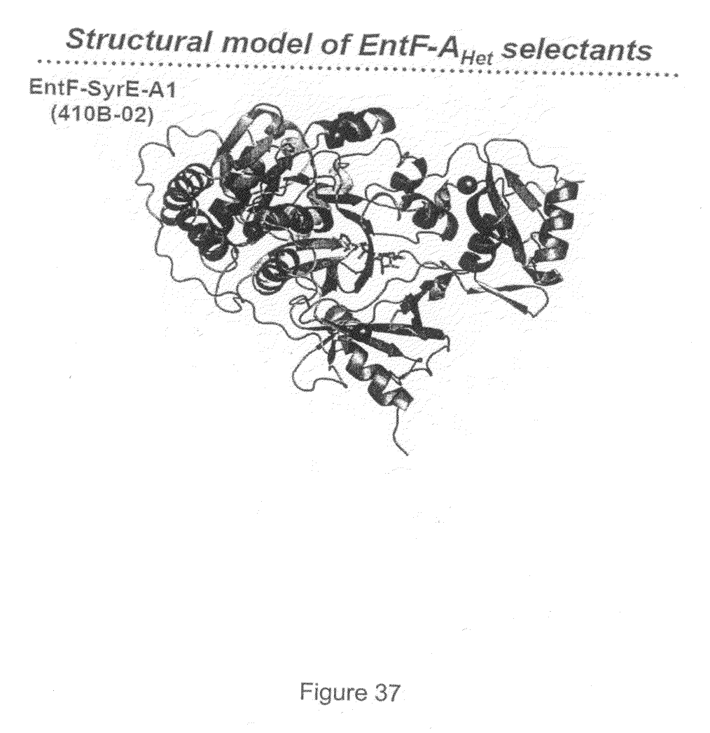

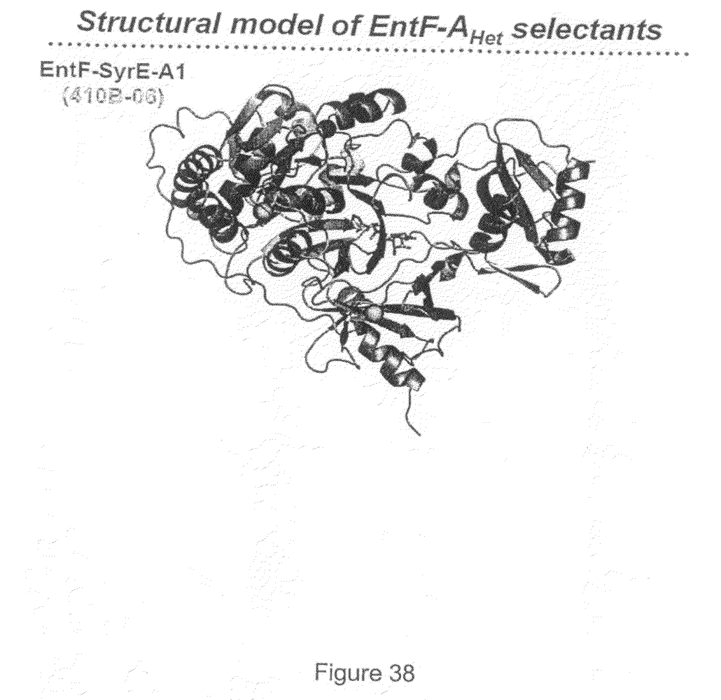

After two rounds of selection, two clones had emerged that had colony diameters similar to that of cells harboring wild-type EntF. These EntF-SyrE-A1 genes from these clones (410B-02 and 410B-06) were isolated and sequenced (FIGS. 33A and 33B), and the proteins encoded by these genes were purified (FIG. 34); both enzymes contain four amino acid substitutions relative to the Round 0 chimera (FIG. 35). The encoded proteins were overexpressed and purified alongside the first round hit (410-H4), the parental (naively-swapped) chimera (410), and wild-type EntF. In a biochemical assay of reconstituted enterobactin biosynthesis in which EntF activity is rate-limiting, one of the second round hits (410B-06) exhibits an 8-fold increase in activity relative to the naively-swapped chimera and is within 4-fold of wild-type EntF activity. While the first round hit (410-H4) does not exhibit a large activity increase relative to the parental chimera, it is much more soluble than the parental chimera, suggesting that both increased protein solubility and activity were achieved with these modified assembly lines. Structural models of the EntF-A_{Het} selectant are shown in FIGS. 36-38. Our results demonstrate that non-specific mutagenesis of an assembly line domain followed by selection for functional biosynthesis by an assembly line containing said domain allows for the generation and isolation of MALs producing functional natural products having altered characteristics. Surprisingly, only two rounds of directed evolution were required to obtain a novel and improved natural product.

### Example 2

### Alterations to the Bacillomycin Assembly Line

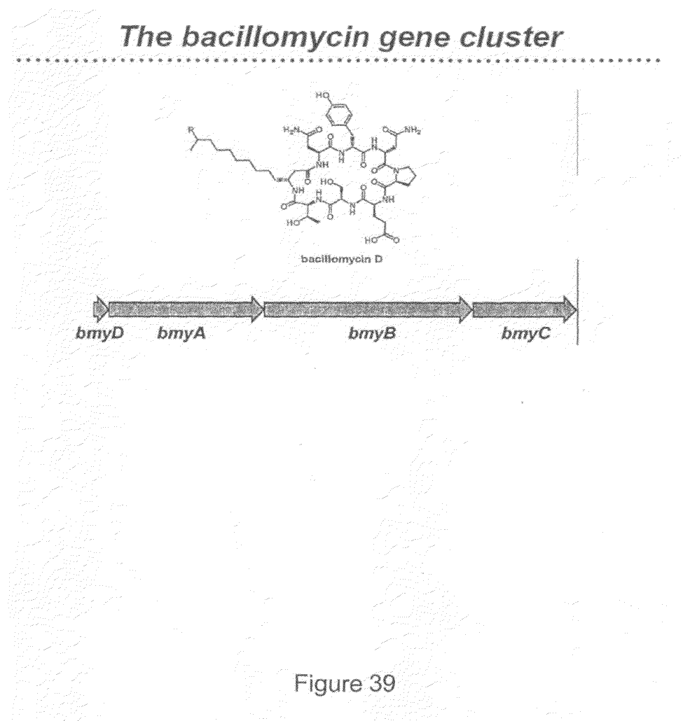

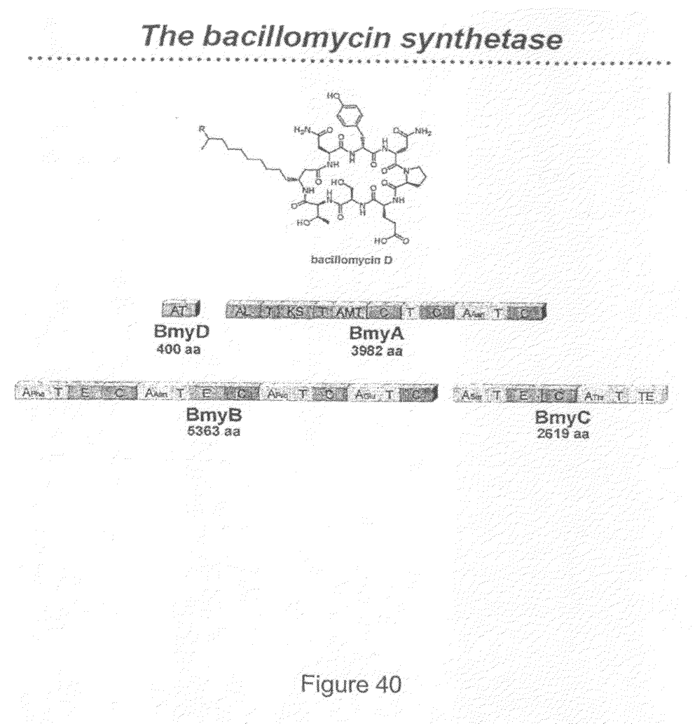

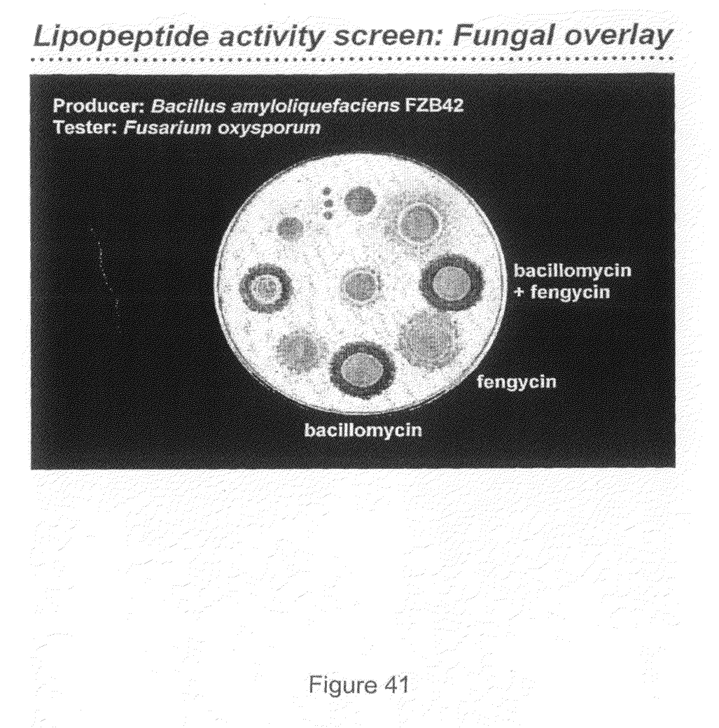

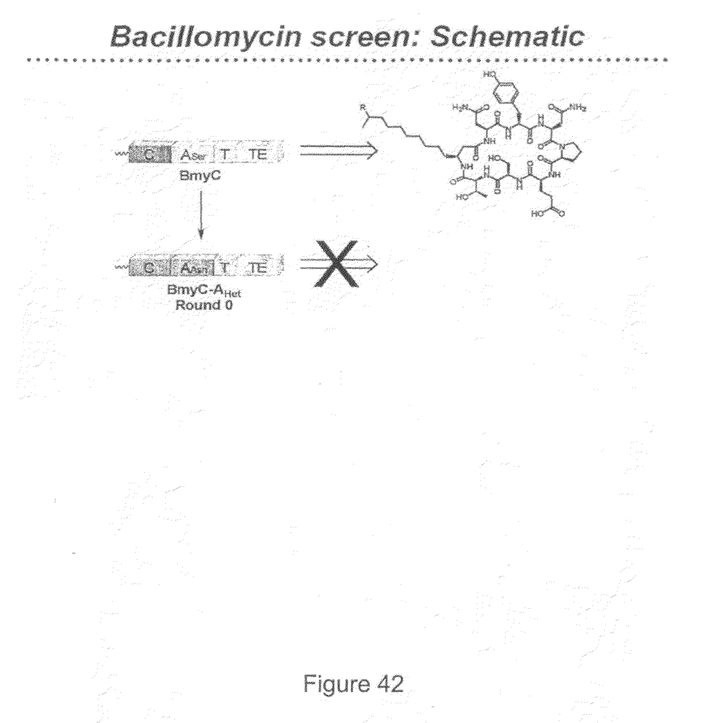

The bacillomycin gene cluster comprises bmyA, bmyB, bmyC, and bmyD (FIG. 39). BmyD is a single AT domain of the initiation module. BmyA contains the remainder of the initiation module and elongation modules; BmyB contains elongation modules; and BmyC contains elongation modules plus the termination TE domain. (FIG. 40). Bacillomycin D activity can be tested by utilizing a screen where the producer Bacillus amyololiquefaciens FZB42 is spread onto a plate containing the fungus Fusarium oxysporum (FIG. 41). Modified assembly lines are generated by replacing the A_{Ser} domain in BmyC with an A_{Asn} domain. This substitution is expected to result in no product being made by the assembly line. By instead substituting mutated A_{Asn} domains into the BmyC gene and selecting for variants with activity in a bacillomycin screen, active variants of the modified assembly line can be identified, where these active, modified assembly lines replace the Ser moiety with an Asn moiety in the resulting product (FIG. 43).

### Example 3

### Mapping Protein-Protein Interactions in the Enterobactin Synthetase

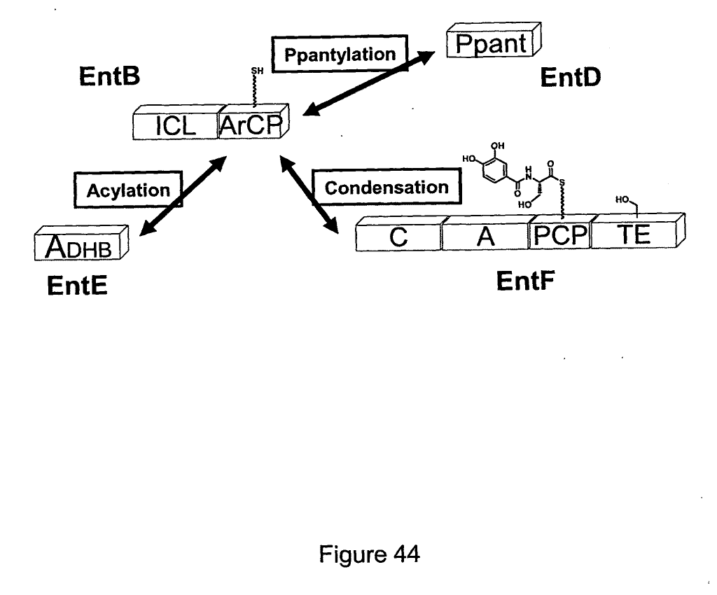

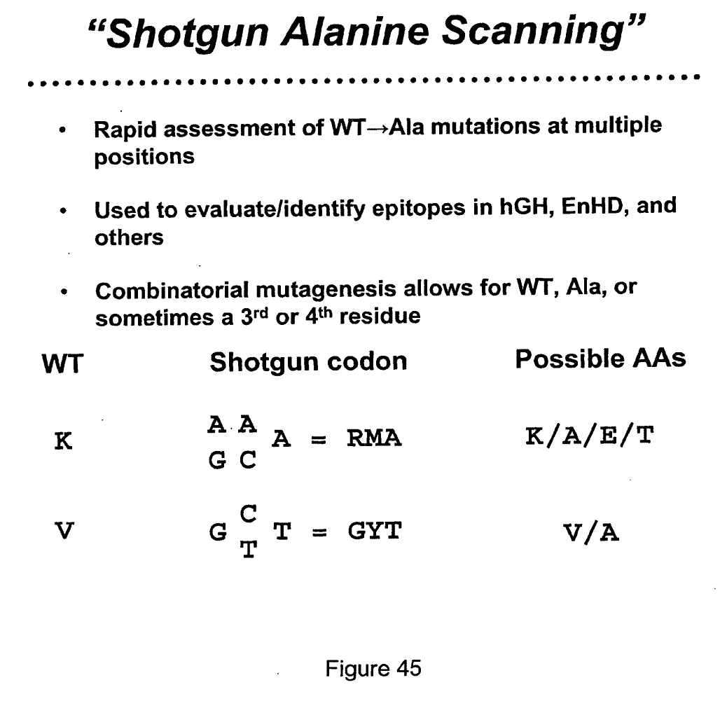

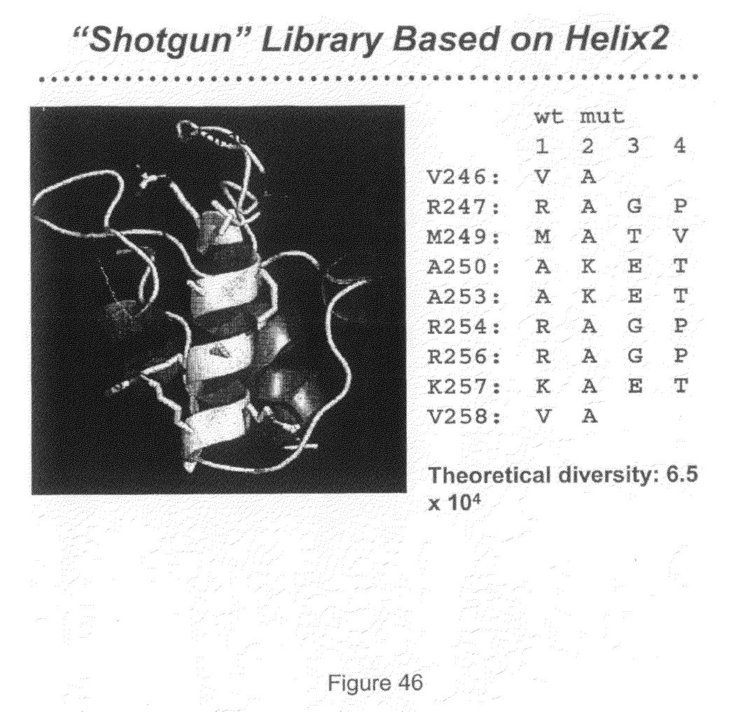

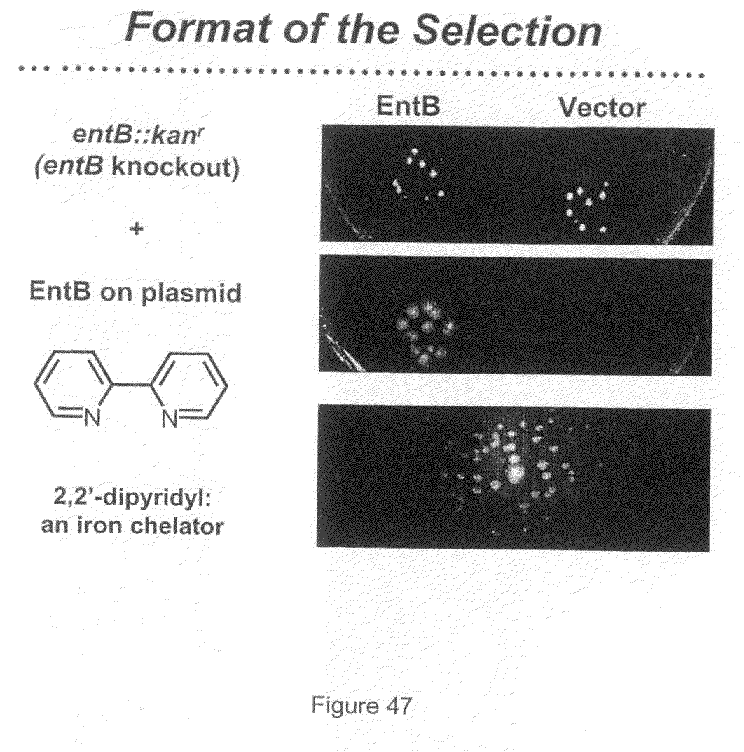

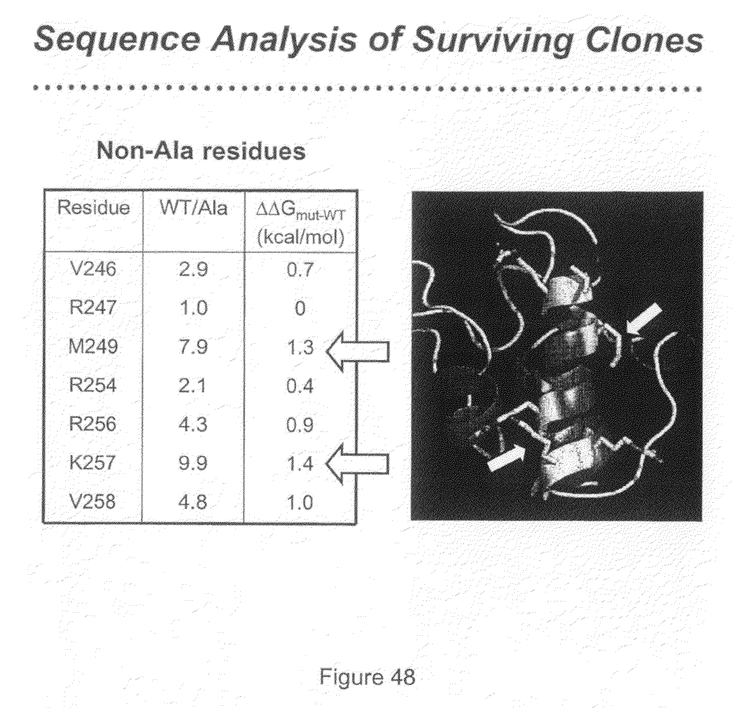

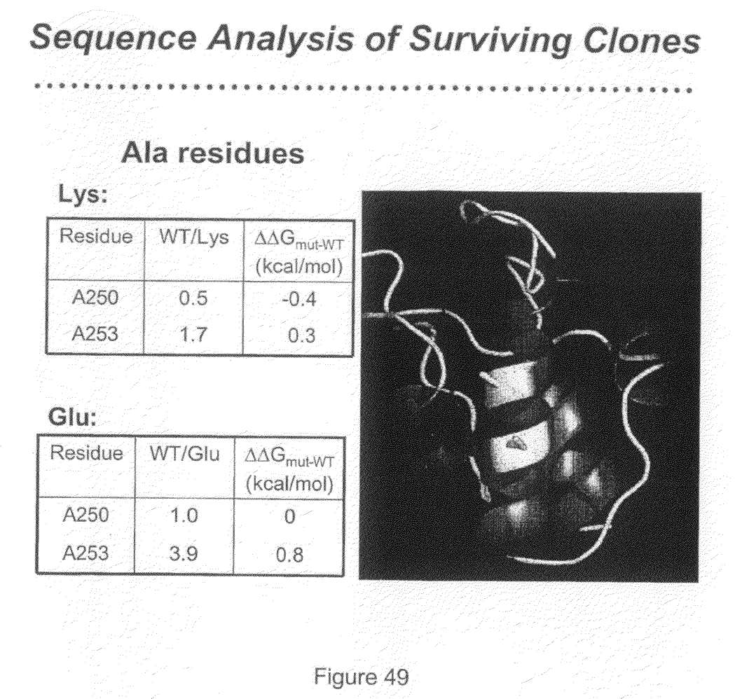

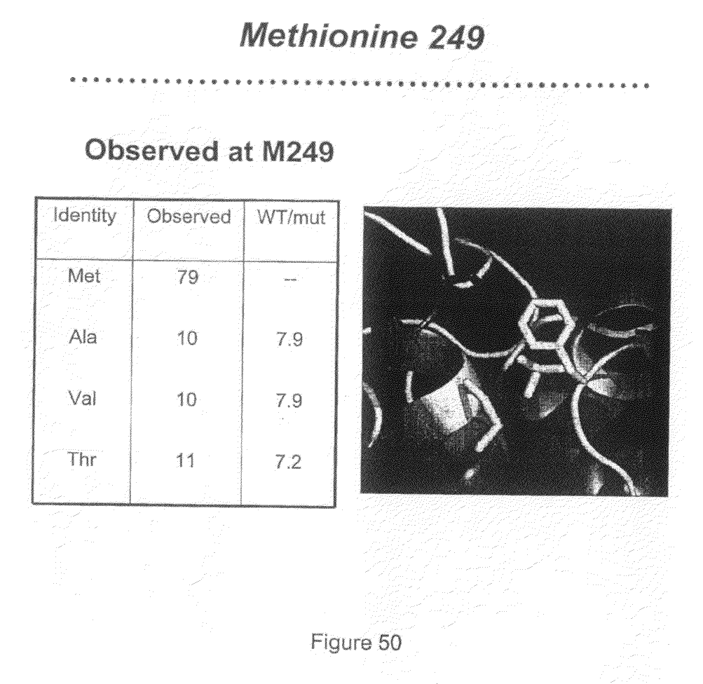

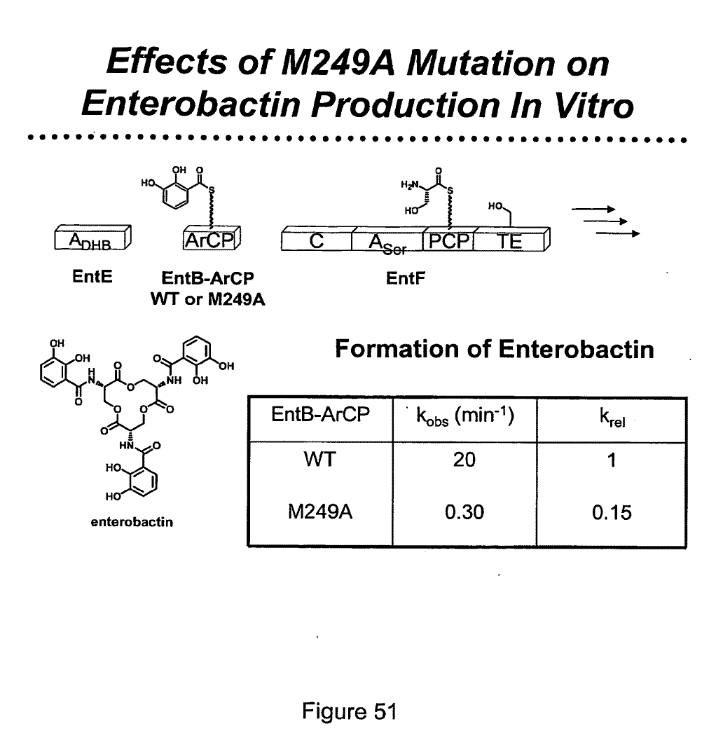

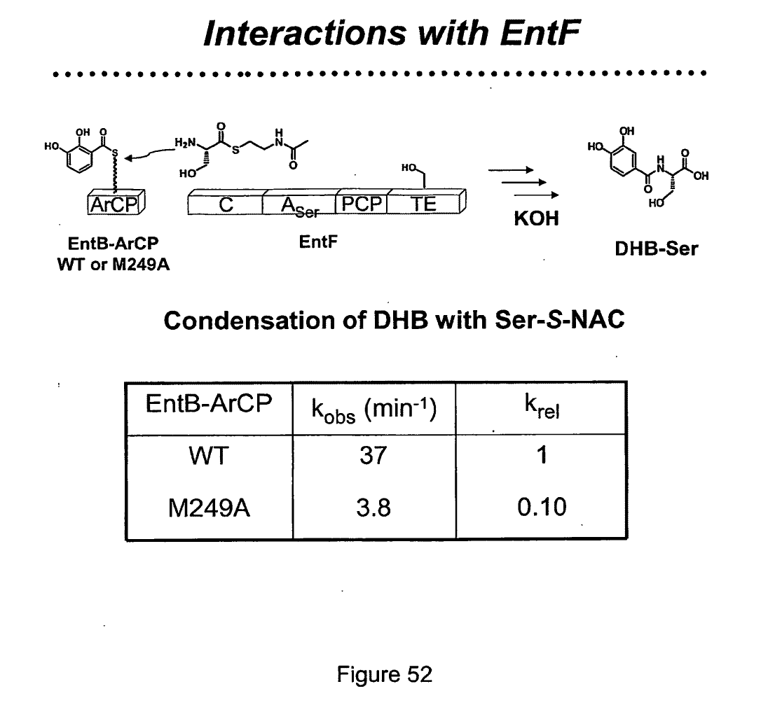

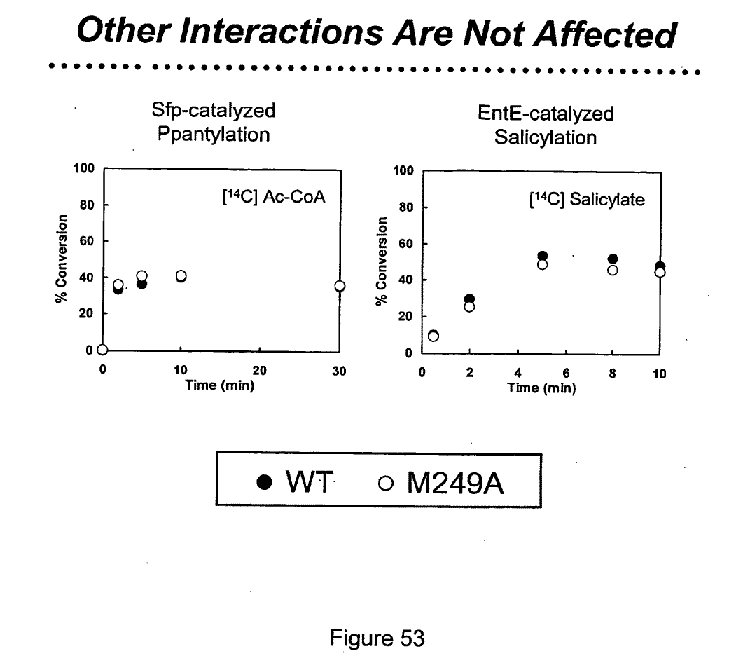

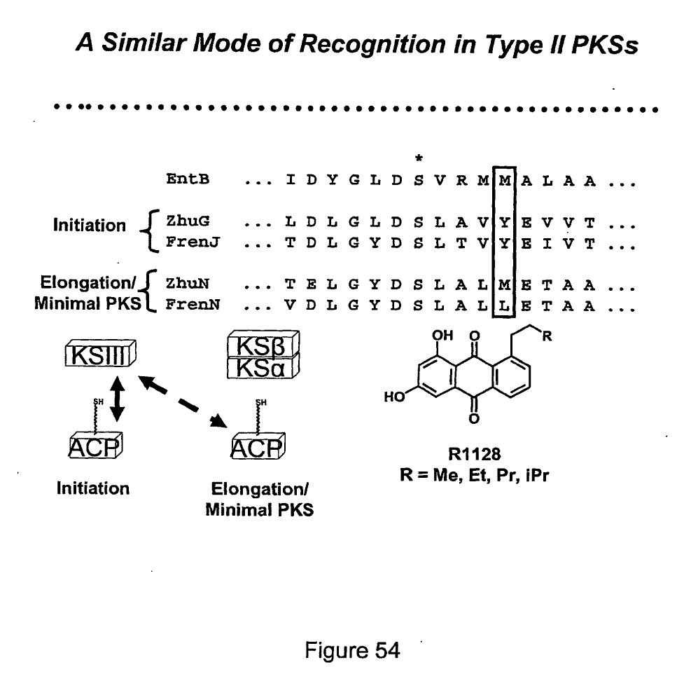

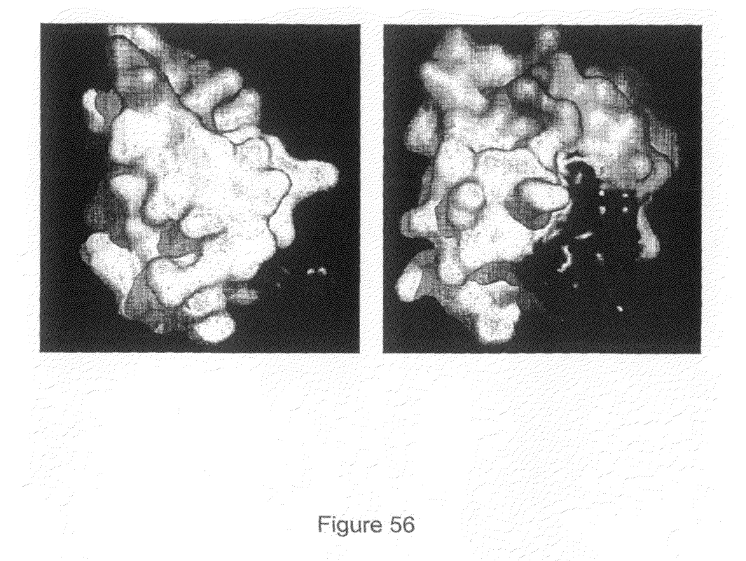

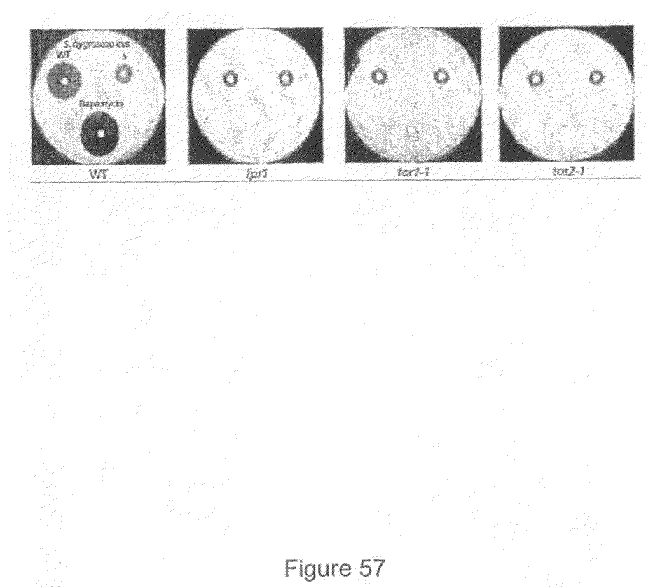

As described above, the enterobactin synthetase assembly line comprises EntB, EntD, EntE, and EntF. Interactions between EntB and the other proteins (EntD, EntE, and EntF) are known to occur (FIG. 44). To assess these interactions, a technique known as “shotgun alanine scanning,” a method known in the art, and described in Weiss et al. ((2000) Proc Natl Acad Sci USA 97, 8950) can be employed. Briefly, this technique allows combinatorial changes at specified residues between WT and alanine (or, for some codons, other amino acids as well). As shown (FIG. 45), this technique allows for rapid assessment of WT→Ala mutations at multiple positions, and has been used to evaluate and identify epitopes in proteins including hGH and EnHD important in specific interactions. To study EntB, shotgun alanine scanning was performed at residues 246, 247, 249, 250, 253, 254, 256, 257, and 258 (FIG. 46) to generate a library with changes at these residues. Using the selection described above, and shown in FIG. 47, ratios of WT to alanine residues among surviving clones was assayed, as used to calculate the energetics of each interaction as detailed in Weiss et al., supra. Particularly important interactions were detected at Met249 and Lys257 (FIG. 48). Also analyzed were changes from alanine at positions 250 and 253 to lysine or glutamate (FIG. 49). The Met249 mutations were further analyzed to determine the ratios methionine to valine or threonine mutations in survival clones. These ratios were observed to be similar to Met249Ala changes (FIG. 50) Next Met249Ala EntB can be shown to to reduces enterobactin production by 85% as compared to WT EntB in vitro (FIG. 51). This mutation also reduced DHB-Ser production as compared to WT by 90% (FIG. 52) based on interactions between EntB and EntF. Other interactions are not affected by this the Met249Ala change, suggesting that this residue is not involved in the Sfp-catalyzed Ppantylation or EntE-catalyzed salicylation (FIG. 53). A similar mode of recognitions may exist in Type II PKSs based on sequence homology (FIG. 54; Tang et al., (2003) Biochemistry 42, 6588). Other shotgun alanine scanning libraries can be created with using other regions of the EntB-ArCP (FIGS. 55 and 56).

Using the above approach, one can modify the protein-protein interactions within an assembly line to enhance biosynthesis or produce novel natural products.

### Example 4

### Localized Protein Interaction Surface on the EntB Carrier Protein Revealed by Combinatorial Mutagenesis and Selection

As substrates for biosynthetic operations are presented on carrier proteins as covalently-attached thioesters (through a 4′-phosphopantetheine cofactor), a detailed understanding of protein-protein interactions between carrier proteins and other domains is required for reprogramming of NRPS/PKS machinery. In this example, we report the identification of a protein interaction surface on the EntB aryl carrier protein (EntB-ArCP) for phosphopantetheinyl transferases (PPTases), such as EntD and Sfp, by combinatorial mutagenesis and selection. This protein interaction surface is highly localized, consisting of just two surface residues, and is distinct from the previously identified interface for the downstream elongation module, EntF.

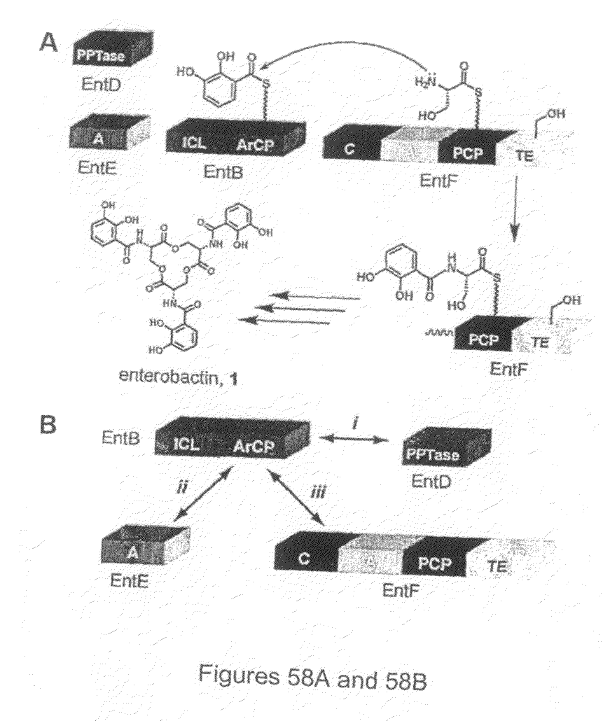

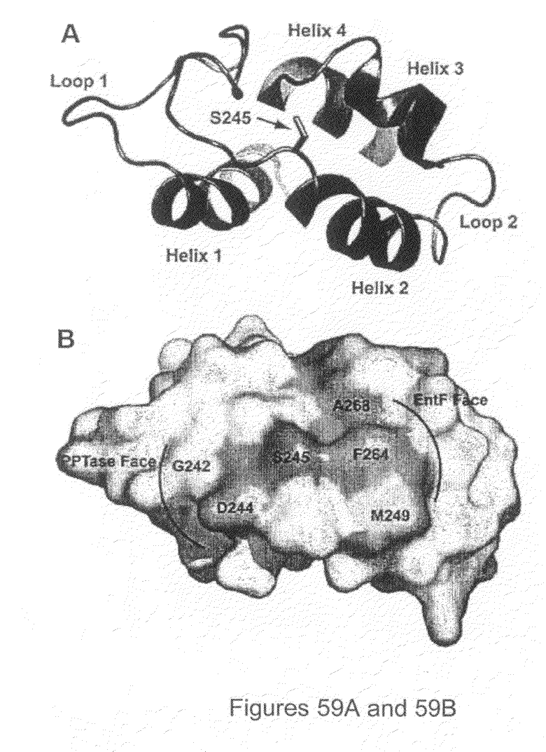

As noted above, enterobactin (1) is an iron-chelating siderophore produced by Escherichia coli upon iron starvation. The enterobactin synthetase consists of four protein components, EntBDEF, that use three molecules each of 2,3-dihydroxybenzoate (DHB) and serine to produce 1 via NRPS logic (FIG. 58A). The ArCP domain of EntB (EntB-ArCP) must participate in three well-timed protein-protein interactions during the biosynthetic reaction cascade (FIG. 58B): (i) with EntD (or other PPTases) during phosphopantetheinylation; (ii) with EntE during activation of DHB and thiolation onto the phosphopantetheine arm of holo-EntB-ArCP; (iii) with EntF during condensation of DHB (presented on the EntB pantetheine) with serine. By using an in vivo selection for EntB function by plating E. coli onto iron-deficient media, we rapidly processed large (>10⁶) EntB mutant libraries for their ability to support production of 1 in vivo. We used this selection together with combinatorial mutagenesis of C-terminal regions of EntB to map an interaction interface on EntB-ArCP for EntF. Using the EntB crystal structure as our guide (FIG. 59A), we designed and prepared three libraries of mutants that collectively span the N-terminal portions of EntB-ArCP: helix 1 (library H1) and the long loop between helix 1 and helix 2 (libraries L1A and L1B). In library H1, non-core residues in helix 1 were allowed to vary between WT and Ala by partial codon variation (due to the degeneracy of the genetic code, a 3^{rd} and 4^{th} residue was permitted at some positions). For libraries L1A and L1B, residues in regions 225-235 and 236-244 (respectively) were subjected to a similar randomization scheme. Selection for clones that produce 1 was then achieved by plating the libraries onto minimal media made iron-deficient by the addition of the metal chelator 2,2′-dipyridyl.

Over 65 non-redundant surviving clones from each library were isolated and sequenced. From these data, WT/Ala ratios for each position, defined as the number of times WT was observed to the number of times Ala was observed, were determined. The degree of conservation for each residue was classified as high (WT/Ala≧20), intermediate (6<WT/Ala<20), or low (WT/Ala≦6). Only five residues fell into the intermediate or high conservation categories (Table 2). FIG. 59B shows the surface of EntB-ArCP color coded according to these classifications, including data compiled from our previous report.

The sequencing results revealed that the residues G242 and D244 form a conserved, surface-exposed patch that immediately precedes the phosphopantetheinylated S245 (FIG. 59B). This cluster corresponds to the interaction surface on EntB-ArCP for PPTases, such as EntD. EntB-ArCP G242A or D244R mutants are poor substrates for EntD. The ArCP mutant D244A is still efficiently phosphopantetheinylated by EntD in vitro, but cannot be recognized by the broad-substrate PPTase Sfp from B. subtilis. Mutation of this conserved Asp, which immediately precedes the phosphopantetheinylated serine, has been reported to disrupt PPTase recognition in EntB and other systems. The interaction surface on EntB-ArCP for PPTase recognition is distinct from that of EntF. Each interaction surface is located on a separate side of S245, and each is comprised of residues from different structural elements. These observations suggest that PPTases and EntF recognize distinct and highly localized interaction faces on EntB-ArCP. Therefore, it should be possible to alter the recognition properties of EntB-ArCP for one of these synthetase components while leaving interactions with the other unaffected.

TABLE 2
WT/Ala ratios for selected residues on EntB-ArCP

| Residue | WT/Ala |
|---|---|
| D234 | 13.7 |
| L238 | >64.0 |
| D244 | 24.0 |
| G242 | 17.8 |
| L243 | 46.0 |

Three other residues displayed intermediate or high conservation: L238, L243, and D234. The residues L238 and L243, located on the loop, point toward the carrier protein core. The high WT/Ala ratios at these positions is likely due to the role of the Leu side-chain in maintaining the stability of the EntBArCP fold. Aspartate at position 234 was preferred about 14-fold over Ala, presumably because D234 participates in charge-charge interactions with K215 and R219 of helix 1.

Collectively, we now have scanned ˜80% of the EntB-ArCP surface using a combinatorial mutagenesis and selection scheme. Overall, the majority of EntB-ArCP surface residues were highly tolerant to mutation. Thirty-six of 44 total surface residues that were examined here and in our earlier report showed low conservation. This result implies that the majority of EntBArCP surface residues are not involved in interactions with other synthetase components.

We and others have found that aryl carrier proteins from EntBDEF and related synthetases are surprisingly impervious to mutation while maintaining their ability to be recognized by free-standing adenylation domains in vitro. Thus, the interface for EntE may be malleable for presentation of aminoacyl-O-AMP to the pantetheinyl arm of EntB.

This example suggest that reprogramming NRPS and PKS assembly lines by engineering selective carrier protein interactions should optimally focus on interaction “hot spots,” similar to those on EntB-ArCP for EntD/Sfp and EntF. This process can be facilitated by directed evolution approaches (e.g., using the methods described herein) that target these regions.

### Library Design and Production

The E. coli K12-derived strain entB::kan^{R} contains a chromosomal replacement of the entB gene with a kanamycin resistance marker. When transformed with a plasmid harboring the entB gene, these cells are able to grow on iron-depleted media. This complementation format allowed us to rapidly process large libraries of EntB variants for function. We used a structural homology model of EntB-ArCP based PCP domain from the tyrocidine synthetase (TycC3-PCP) for our analysis. A crystal structure of full-length EntB (apo-form) (Drake et al., Chem. Biol. 13:409-419, 2006), which we used for our subsequent library design

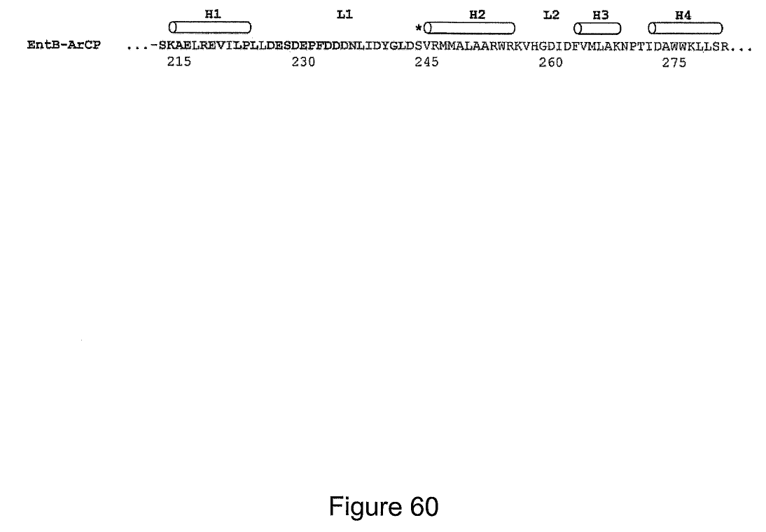

Three shotgun alanine scanning libraries that span helix 1 (library H1) and the long loop between helix 1 and helix 2 (loop 1, libraries L1A and L1B) were constructed as described below. (Regions of EntB-ArCP corresponding to helices 2 and 3 and loop 2 were examined as described herein.) FIG. 60 shows the sequence of EntBArCP from E. coli CFT073, with the positions of randomization for each library indicated. The shotgun alanine scanning randomization scheme allows residues to vary between WT, Ala and in some cases a 3^{rd} or 4^{th} residue. Position 216, in which the WT residue is Ala, was allowed to vary between Ala (WT), Lys, Glu, and Thr. For all three libraries, the theoretical diversity (8×10³ for H1, 4×10¹³ for L1A, and 1.6×10⁴ for L1B) was well represented among the total library clones (6.7×10⁴ for H1, 5×10⁸ for L1A, and 5×10⁷ for L1B).

Library Selection and Functional Mapping onto the EntB Crystal Structure

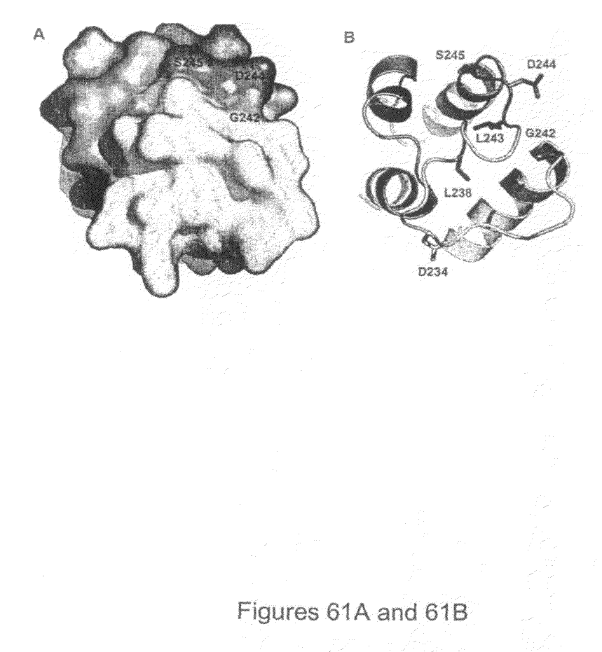

Selection for functional EntB variants was achieved by plating the libraries onto minimal media made iron-deficient by the addition of 100 μM 2,2′-dipyridyl. After incubation at 37° C. for two overnights, colonies of varying diameters were observed The largest colonies were picked, restreaked onto selective media, and sequenced. Table 3 contains the compiled data from sequencing of 69, 88, and 75 nonredundant clones from the surviving pools of H1, L1A and L1B, respectively. For each position, the WT/Ala ratio was used as a measure of conservation, where the WT/Ala ratio is defined as the number of times WT side-chain identity was observed to the number of times Ala was observed. For position 216 (in which Ala is the WT residue), the WT/Lys ratio was used. The degree of conservation at each position was categorized as high (WT/Ala≧20), intermediate (6<WT/Ala<20), or low (WT≦6). The surface representation of the apo-EntB-ArCP crystal structure, where each position is color coded according to these classifications, is shown in FIGS. 61A and 61B.

TABLE 3

| Residue | WT/Ala | WT/mut2a | WT/mut3a |
|---|---|---|---|
| Surviving clones from | | | |
| library H1b | | | |
| S214 | 2.1 | — | — |
| K215 | 3.8 | 6.4 (E) | 9.0 (T) |
| A216 | WT/Lys = 0.5 c | 0.7 (E) | 1.6 (T) |
| E217 | 1.2 | — | — |
| R219 | 2.5 | 2.9 (G) | 12.7 (P) |
| E220 | 1.1 | — | — |
| V221 | 1.8 | — | — |
| L223 | 2 | 3.4 (V) | 18.5 (P) |
| P224 | 0.3 | — | — |
| Surviving clones from | | | |
| library L1Ad | | | |
| L225 | 1.1 | 1.1 (V) | 2.4 (P) |
| D227 | 2 | — | — |
| E228 | 3.2 | — | — |
| S229 | 0.8 | — | — |
| D230 | 3.4 | — | — |
| E231 | 2 | — | — |
| P232 | 1.9 | — | — |
| F233 | 1.4 | 0.8 (S) | 2.2 (V) |
| D234 | 13.7 | — | — |
| D235 | 5.8 | — | — |
| Surviving clones from | | | |
| ibrary L1Be | | | |
| D236 | 1.3 | — | — |
| N237 | 6.2 | 0.9 (D) | 10.3 (T) |
| L238 | >64.0 | 6.4 (V) | 64.0 (P) |
| I239 | 1.5 | 0.8 (T) | 1.2 (V) |
| D240 | 2.6 | — | — |
| Y241 | 2.8 | 44.0 (D) | 3.1 (S) |
| G242 | 17.8 | — | — |
| L243 | 46 | 1.6 (V) | >46.0 (P) |
| D244 | 24 | — | — |

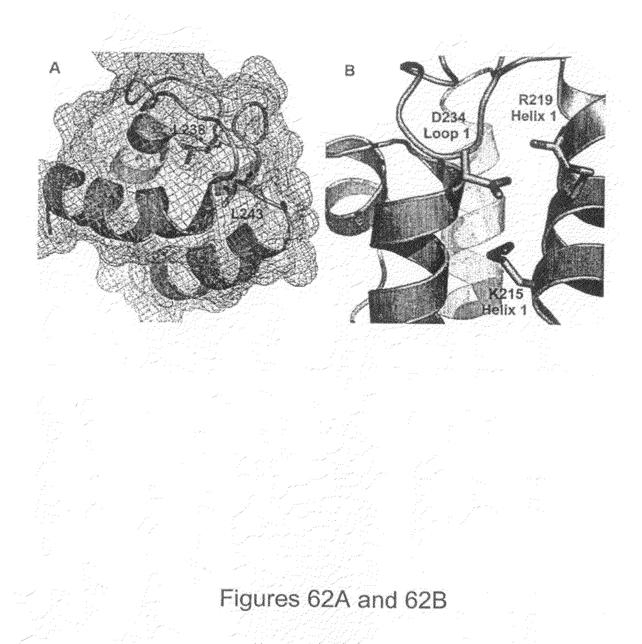

Several positions fell into the high or intermediate conservation category. The sidechains for L238 and L243 point toward the core of the ArCP domain (FIG. 62A) and are likely involved in maintaining the stability of the ArCP fold. The residue D234 is located on loop 1 and appears to participate in charge-charge interactions with K215 and R219 of helix 1 (FIG. 62B). A patch of conserved surface-exposed residues immediately preceding the phosphopantetheinylated S245 consists of G242 and D244. These residues constitute the interaction surface for EntD and Sfp.

### In Vitro Characterization of G242A

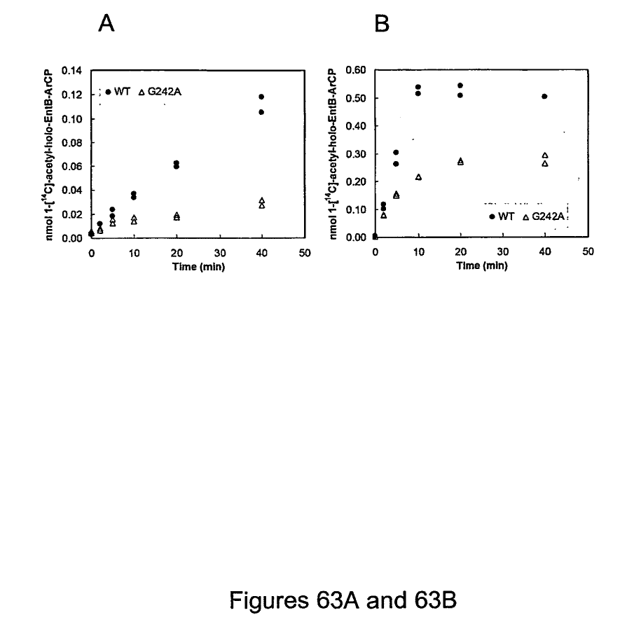

We prepared a variant of the EntB ArCP domain containing a G242A mutation. The ability of EntD and Sfp to recognize and efficiently phosphopantetheinylate this mutant was examined by monitoring incorporation of 1-[¹⁴C]-acetyl-CoA onto the ArCP over time. FIGS. 63A and 63B show the time course for phosphopantetheinylation for WT and G242A ArCPs (15 μM) by EntD (5 μM) and Sfp (300 nM). In both cases, G242A was phosphopantetheinylated to a much lower degree than WT ArCP. These results were confirmed by an HPLC assay using coenzyme A (CoASH) as the substrate. Overnight incubation of G242A with Sfp and CoASH resulted in <50% conversion to the holo-form.

### In Vitro Characterization of D244A and D244R

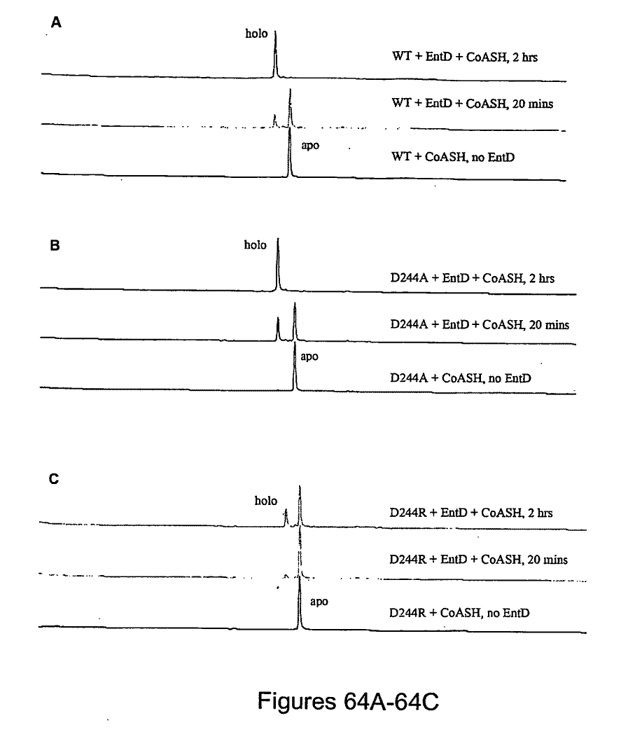

In order to determine the role of D244, we expressed and purified the ArCP mutants D244A and D244R. Using an HPLC assay, D244A was be readily converted to the holo-form by EntD, but not by Sfp (FIG. 64). However, the D244R mutant could not be efficiently phosphopantetheinylated past 50% after 2 hours incubation with EntD and CoASH (conditions sufficient to result in 100% conversion to the holo-form in WT EntBArCP). Gulick and coworkers previously reported that the same mutation (D244R) resulted in an EntB variant that could be converted to only ˜35% holoform using in vivo expression conditions that gave 100% holo-form for WT and several other EntB mutants (Drake et al., Chem. Biol. 13:409-419, 2006). Thus, D244 is involved in recognition by both EntD and Sfp PPTases. In our in vivo selection, the residue at position 244 can vary between WT (Asp) and Ala. The high WT/Ala ratio observed, despite the fact that D244A is still efficiently phosphopantetheinylated by EntD in vitro, suggests that other PPTases may play a role in modification of EntB-ArCP in vivo.

### Library Production and Selection

The plasmid pJRL16 contains the entB gene cloned into a pET22b-based plasmid. For each library, an inactive template based on pJRL16 was produced that contained two sequential TAA stop codons and a unique EcoRI site in the region of entB to be randomized. The appropriate inactive template was used for full plasmid replication with the phosphorylated primers 5′-CCA GCA CCT ATC CCC GCC KCC RMA RMA GMA CTG SST GMA GYT ATC SYT SCA TTG CTG GAC GAG TCC GAT-3′ for H1, 5′-GAG GTG ATC CTG CCG SYT CTG GMT GMA KCC GMT GMA SCA KYT GMT GMT GAC AAC CTG ATC GAC-3′ for L1A, or 5′-GAA CCC TTC GAT GAC GMT RMC SYT RYT GMT KMT GST SYT GMT TCG GTG CGC ATG ATG GCG-3′ for L1B (regions of randomization indicated in bold, hybridization regions indicated in italic; the standard abbreviations for DNA degeneracies are used: K=G/T, M=A/C, R=A/G, S=G/C, Y=C/T. Ligation of the nascent DNA was accomplished by addition of Taq ligase to the reaction mixture. Plasmid replication with the library primers resulted in replacement of the stop codons S and the EcoRI site with the desired regions of randomization. The template was then destroyed by double digestion with DpnI and EcoRI.; and the library DNA was purified by phenol/chloroform extraction. Transformation of library DNA into entB::kan^{R} cells was achieved by electroporation. In a typical selection b 10⁴-10⁷ cells were plated onto 241−×241 -mm plates of minimal media containing 100 μM 2,2′-dipyridyl and 100 μg/mL carbenicillin, and grown for two overnights at 37° C. The largest colonies were restreaked onto selective media and sequenced.

### Purification and Characterization of G242A, D244A, and D244R.

The DNA for G242A, D244A, and D244R was prepared using standard methods. Expression and purification of EntB-ArCP (WT and mutants) and EntD was as previously described. Phosphopantetheinylation assays monitored by radioactivity were performed in 75 mM Tris pH 7.5, 10 mM MgCl₂, 0.5 mM TCEP using 69 μM 1-[¹⁴C]-acetyl-CoA (6.6 Ci/mol) and 15 μM EntB-ArCP (WT or mutants). The total reaction volume was 50 μL. Reactions were initiated by addition of EntD or Sfp and quenched in 500 μL 10% (w/v) trichloroacetic acid (TCA). The protein pellet was recovered by centrifugation, washed with 10% (w/v) TCA, and then redissolved in 100 μL formic acid. Scintillation fluid was added (4 mL) and the amount of incorporated radiolabel was determined by liquid scintillation counting. Conditions for the HPLC phosphopantetheinylation assay were similar. Following incubation with CoASH (5 mM) and EntD or Sfp, reactions were quenched in water/0.1% TFA. Analysis was performed using a C4 HPLC column with water/0.1% TFA and acetonitrile as the mobile phases.

### Example 5

### Interdomain Communication Studied by Combinatorial Mutagenesis and Selection

To assess surface features of the EntF T domain recognized by C, A, and TE, regions of the EntF T domain were submitted to shotgun alanine scanning and Ent production selection, which revealed residues that could not be substituted by Ala. EntF mutants bearing Ala in such positions were assayed in vitro for Ent production with EntEB and A-T, C-T, and T-TE communications. From these studies, G1027A and M1030A were found to be specifically defective in acyl transfer from T to TE. Thus, these mutants define an interaction surface between these two in cis domains in an NRPS module.

In the two-module EntEBF system EntEB acts as initiation module, while EntF functions as both an elongation and a termination module. Given that the four-helix T domain scaffolds can be distinguished, at least by some partner proteins that work in trans, we sought to determine if the EntB T domain presents different faces to its distinct partners, EntD (the PPTase), EntE (the A domain), and EntF (C domain). To do so, we employed a selection under low iron conditions where E. coli require the capacity to produce enterobactin to grow on low iron media. By combinatorial mutagenesis of selected regions on EntB, we identified a surface of the EntB T domain that, upon mutation in the comprising residues, was specifically impaired for recognition by the EntF elongation module but not interaction with EntD or EntE. In this example, we have turned to the in cis T domain of the 142 kDa protein EntF to assess comparable libraries by combinatorial mutagenesis.

### Homology Modeling of the EntF T Domain and Library Design

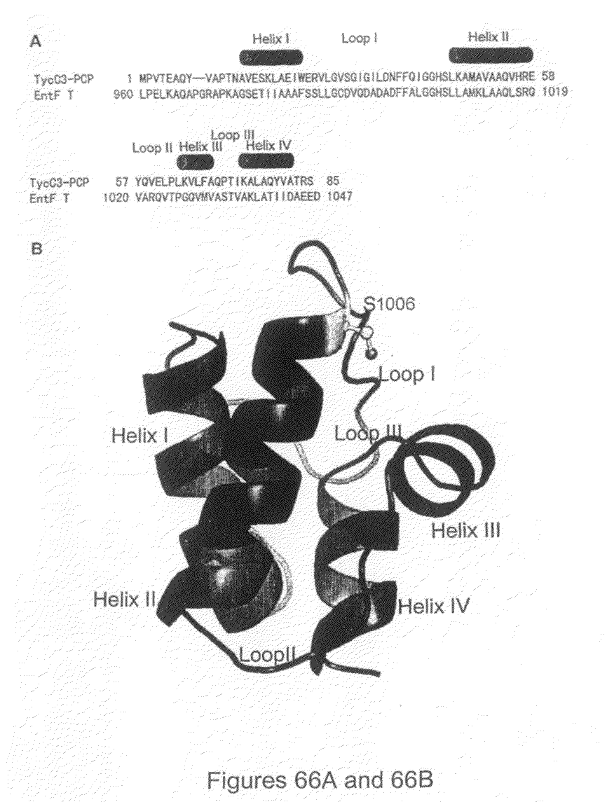

Carrier protein domains are approximately 80 to 100 residues in length. A structure of the EntF T domain is not currently available; we therefore produced a structural model based on homology with a T domain from the tyrocidine NRPS system (TycC3-PCP). Residues 960-1047 of EntF were aligned with TycC3-PCP by using the ClustalW algorithm (FIG. 66A). The EntF T domain shares 30% sequence identity with TycC3-PCP (this value is higher than the sequence identity that the EntF T domain shares with the EntB T domain), which suggests that these two carrier proteins should have similar folds. Indeed, several carrier proteins from primary or secondary metabolism have been shown to adopt three or four-helix bundle structures similar to that of TycC3-PCP. Using the TycC3-PCP NMR structure as template, we generated a structural model of the EntF T domain, shown in FIG. 66B. As with other carrier proteins, our EntF T domain homology model comprises a four-helix bundle structure. A long loop links helix I and helix II, a short loop connects helix II and helix m, and an even shorter loop is found between helix III and helix IV. The site of phosphopantetheinylation, Ser1006, is located at the N-terminal end of helix II.

Helix II of the B. subtilis ACP from primary metabolism has been reported to be important for interaction of the ACP with its cognate phosphopantetheinyl transferase ACPS (ACP synthase). Also, helix II residues on PCPs have been reported for interaction with catalytic partners. Residues in helix III of EntB-ArCP constitute an interaction interface for the downstream elongation module, EntF. Therefore, we targeted these portions on the EntF T domain surface (predicted to lie in the helix II/loopII/helixIII region) for combinatorial mutagenesis via shotgun alanine scanning. In this combinatorial mutagenesis strategy, codons are used that allow the residues to vary between wt, Ala, and sometimes a third or fourth residue. For cases where the wt residue was Ala, we used a combinatorial codon set that allowed the side-chain identity to vary between Ala, Glu, Gln, Pro. Three libraries spanning regions of helix II, helix III, and loop II/helix III were prepared (FIGS. 66A and 66B). The theoretical sequence diversity for the three libraries (1024, 256, and 256 for helix II, helix III, and loopII/helixIII, respectively) were adequately represented among our total clones for each library (2×10³ for helix II, 1×10³ for helix III, and 5×10² for loopII/helix III).

### In Vivo Selection for Enterobactin Production

Selection for functional EntF clones was based on the fact that enterobactin production is essential for the survival of E. coli under low iron conditions. The E. coli strain entF::cat (ER 1100A) contains a chromosomal replacement of the entF gene by a chloramphenicol resistance marker. The entF::cat strain is not able to grow in minimal media in which iron is sequestered by the chelator 2,20-dipyridyl. However, the entF knockout cells can be complemented by transformation with a pET29-based plasmid that harbors the wild-type entF gene.

Bacteria harboring the EntF libraries were subjected to the iron-deficient selection conditions. Colonies of varying sizes were observed after 24 hr at 37° C., the largest of which were isolated and sequenced. Twenty-nine, 16, and 17 nonredundant surviving clones from the helix II, helix III, and loop II/helix III libraries (respectively) were analyzed. Further sequencing of survival colonies from helix III and loop II/helix III (40 and 44 total colonies sequenced, respectively) yielded redundant sequences. This result might be due to the small sequence diversity of these two libraries. The survival rate on selection medium was estimated by comparing numbers of colonies that grew on rich media with the number of colonies that grew on low-iron media. We observed survival rates of 30% for the helix II library, 8% for the helix III library, and 15% for the loop II/helix III library.

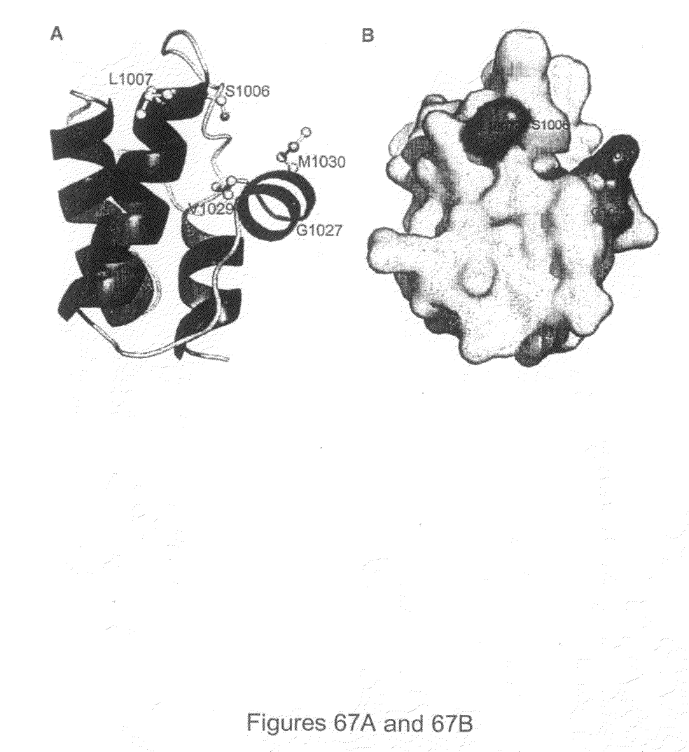

For residues L1007, G1027, V1029, and M1030, the wt amino acid was strongly preferred over Ala (no Ala residues were observed in surviving clones at these positions. The residue V1029 is predicted to be a core residue in the EntF T domain homology model; furthermore, NMR studies of an EntF fragment confirmed that V1029 points toward the core of the EntF T domain (D. Frueh, D. Vosburg, C. T. W., G. Wagner, unpublished data). We therefore reasoned that mutation of V1029 would be likely to cause disruption of the EntF T domain structure, and thus we did not characterize any point mutants at this position. Residue L1007 is located on helix II of the EntF T domain homology model, immediately C-terminal to the phosphopantetheinylated Ser. The analogous position was found to be important for interactions between the PPTase and ACP of the B. subtilis FAS. Therefore, we believe that mutation of L1007 affect posttranslational modification of EntF. The residues G1027 and M1030 lie on helix III of the EntF T domain model. A representation of the EntF T domain homology model with the locations of the conserved residues is shown in FIGS. 67A and 67B. Based on the above sequence analysis, we expressed and purified wild-type EntF along with several variants that contained single mutations in the T domain: L1007A, G1027A, and M1030A. Expression of EntF (wt and mutants) and the other synthetase components proceeded in good yield and purity by using established protocols.

From the sequencing results for the survivors, proline was prohibited within a-helical regions, except at the beginning of helix III. Proline is an α-helix-breaking residue and would likely disrupt the structure of the EntF T domain if placed in the middle of an α-helix. The observation that proline was not observed in α-helical positions of the EntF T domain (where proline was permitted as an option) suggests that E. coli survival under low iron conditions is tightly coupled to EntF function. Under low iron conditions, E. coli thus are under selective pressure for well-folded and functional EntF variants. This result therefore confirms that the information from sequencing results is valuable for dissecting EntF function.

Phosphopantetheinylation Assay

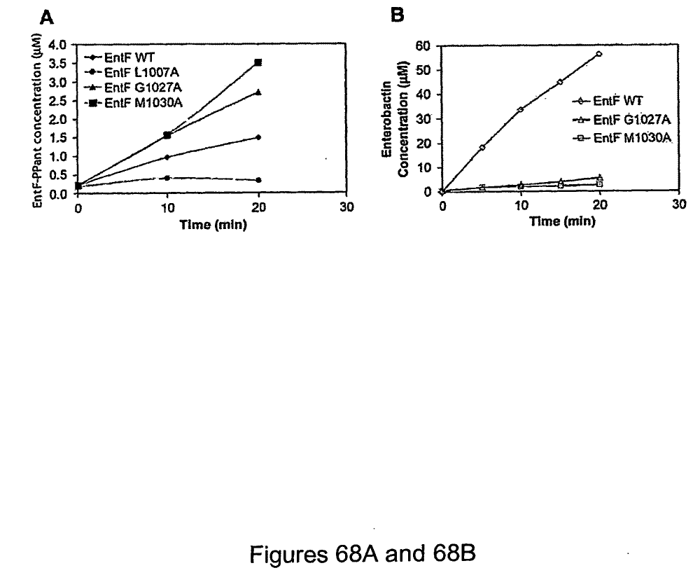

Enterobactin production by the Ent synthetase requires that the T domains of EntB and EntF be primed with the 40-phosphopantetheine prosthetic group. Two endogenous PPTases are found in E. coli: one for primary metabolism (ACPS) and the dedicated PPTase EntD, which is encoded in the enterobactin biosynthetic gene cluster. The PPTase ACPS is responsible for the modification of ACP for fatty acid synthesis but does not accept the EntF T domain as a substrate. However, expression of EntD is upregulated in response to low iron conditions, resulting in the posttranslational modification of the EntB and EntF T domains to their holo forms. In order to determine whether the observed conservation of L1007, G1027, and M1030 during in vivo enterobactin production selection was due to recognition defects between EntF and EntD, a phosphopantetheinylation assay was performed with EntD and EntF (wt and mutants). FIG. 68A shows the initial rate of radiolabeled [³H] coenzyme A incorporation into apo-EntF and mutants catalyzed by EntD. WT EntF and the T domain mutants G1027A and M1030A were phosphopantetheinylated by EntD. Surprisingly, these two mutants (G1027A and M1030A) were phosphopantetheinylated at a slightly higher rate than wt EntF. The reason for this elevated rate of phosphopantetheinylation is not clear. However, the apo-EntF L1007A mutant was not accepted as a substrate for EntD, suggesting that L1007, located immediately adjacent to the phosphopantetheinylated serine in the homology model, is important for recognition by EntD. Furthermore, L1007A could not be recognized by the broad-substrate PPTase Sfp from B. subtilis (data not shown). The defect in recognition of L1007A by EntD rationalizes the observed conservation of wt side chain identity in the in vivo selection. Interestingly, the aligning residue of the ACP from the B. subtilis FAS has been shown to be important for recognition by its cognate PPTase ACPS. As L1007A could not be phosphopantetheinylated (and therefore could not be converted to the active form), we did not pursue further biochemical characterization of this mutant. However, both G1027A and M1030A could be efficiently recognized by EntD, indicating that the conservation of these residues was not due to the participation of these residues in interactions with EntD.

### In Vitro Reconstitution of Enterobactin Biosynthesis

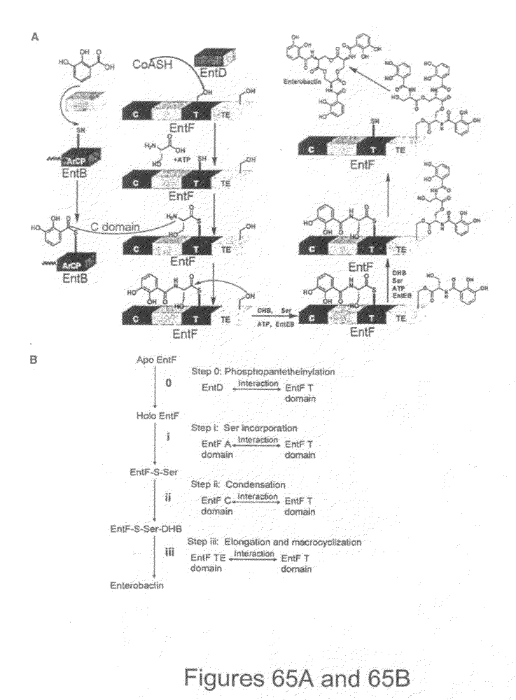

We characterized the mutants G1027A and M1030A in a previously reported enterobactin reconstitution assay involving EntE and EntB Gehring et al., Biochemistry 37:2648-2659, 1998. This assay allows validation of the sequence results from combinatorial mutagenesis and affords the opportunity to quantitatively evaluate the overall competence of the EntF mutants for the three steps of the enterobactin biosynthesis reaction cascade (shown in FIG. 65B). These three steps are: (1) Ser loading, (2) condensation of Ser with DHB (each substrate is tethered to the appropriate T domain), and (3) elongation and macrocyclization. The three reactions are directed by in cis interactions between EntF domains (A-T for loading, C-T for condensation, and T-TE for elongation and macrocyclization).

To prepare the holo form of EntF (wt and mutants), we used the broad-substrate PPTase Sfp from B. subtilis. Both mutants could be efficiently phosphopantetheinylated by Sfp. As the Kₘ for DHB-SEntB-ArCP as the substrate of EntF is approximately 1 μM, reconstitution assays were preformed at 15 μM EntB-ArCP so that catalysis involving EntF would be the rate-limiting step in enterobactin production. This condition allowed us to evaluate whether the EntF mutants were deficient in any of the in cis interactions listed above.

The production of enterobactin is shown in FIG. 68B. The mutants G1027A and M1030A had lower initial rates of enterobactin production than wt EntF, by 15- and 30-fold, respectively, confirming that mutation of these two residues deleteriously affects the enterobactin synthetase, which correlates with the observed sequence conservation data. As both G1027A and M1030A could be phosphopantetheinylated by Sfp (used in this assay), the precise mechanism for the defect in enterobactin production displayed by these mutants must be due to deficiencies in the (1) Ser incorporation step, (2) the condensation step, (3) the elongation and macrocyclization step, or combinations thereof. Each of these steps requires a separate interdomain communication event with the in cis EntF T domain (FIG. 65B). Thus, these mutations affect in cis interdomain interactions such that they hinder function of the EntF module.

Loading of Ser onto the EntF T Domain

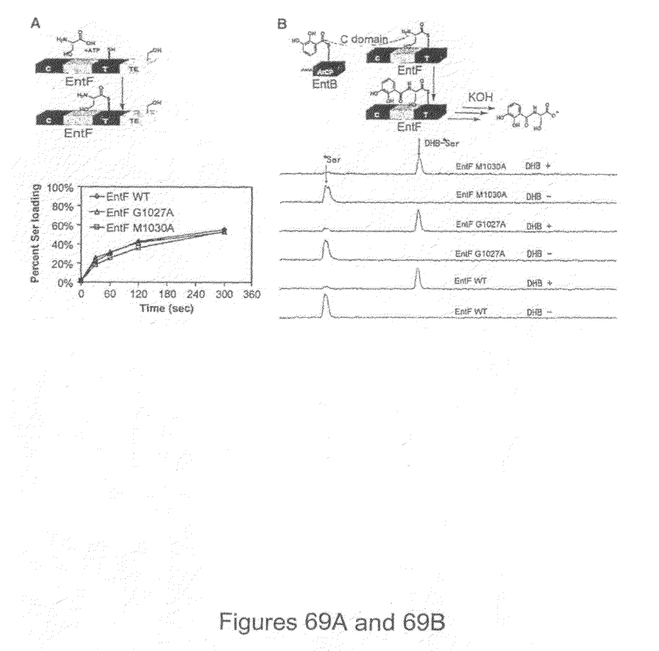

The loading of Ser onto EntF T domain by the EntF A domain is a two-step process. First, Ser is adenylated by the A domain to form the activated Ser-O-AMP ester. Second, this activated Ser-O-AMP species is coupled to the thiol on the phosphopentetheinyl arm of the EntF T domain. To examine the kinetics of Ser covalent loading onto the EntF T domain, the time course for loading of ¹⁴C labeled serine was determined (FIG. 69A). For wt EntF, rapid incorporation of radiolabeled serine was observed within 5 min. Neither G1027A nor M1030A displayed a significant difference in the rate of serine loading relative to wt EntF. These observations suggest that the G1027A and M1030A mutations do not disrupt enterobactin synthesis at the serine loading step. These residues are thus not involved in communication between EntF T domain and the EntF A domain.

### Condensation Assay

Following the loading of serine onto the EntF T domain, the C domain of EntF catalyzes the condensation of DHB (loaded on EntB-ArCP) with the serine loaded on the EntF T domain to form a DHB-Ser condensation product (FIG. 69B). In order to compare the ability of the EntF wt and mutants to perform the condensation between DHB (presented on EntB) and Ser without artifacts arising from transfer of DHB-Ser to the adjacent TE domain or release of DHB-Ser by TE, we produced the EntF C-A-T tridomain proteins for wt, G1027A, M1030A. These C-A-T constructs lack the TE domain, and therefore condensation should be a single turnover event with DHB-Ser accumulating as the covalently bound thioester on the T domain phosphopantetheine group. In order to facilitate detection, radiolabeled [¹⁴C] Ser was employed; reaction products tethered to the T domain of EntF (wt and mutants) were released by treatment with KOH and analyzed on an HPLC equipped with tandem UV and radioactivity detectors. As shown in FIG. 69B, complete conversion from of Ser to DHBSer was observed for wt and both mutants within 15 s of initiation of the reaction by adding DHB. Thus, condensation is a rapid process, and the EntF mutants G1027A and M1030A are not deficient in their ability to catalyze condensation between DHB and Ser. These results indicate the in cis communication between the EntF T domain and the EntF C domain is not adversely affected by mutation of G1027 and M1030. There is no biochemical evidence that suggests that the in trans interaction between EntB and EntF involves any portion of the EntF T domain. The results of the DHB-Ser condensation assay with the C-A-T tridomains for both wt and mutants show that these mutations do not affect any possible interaction between the EntF T domain and EntB.

Acyl-Transfer from the T Domain to the TE Domain

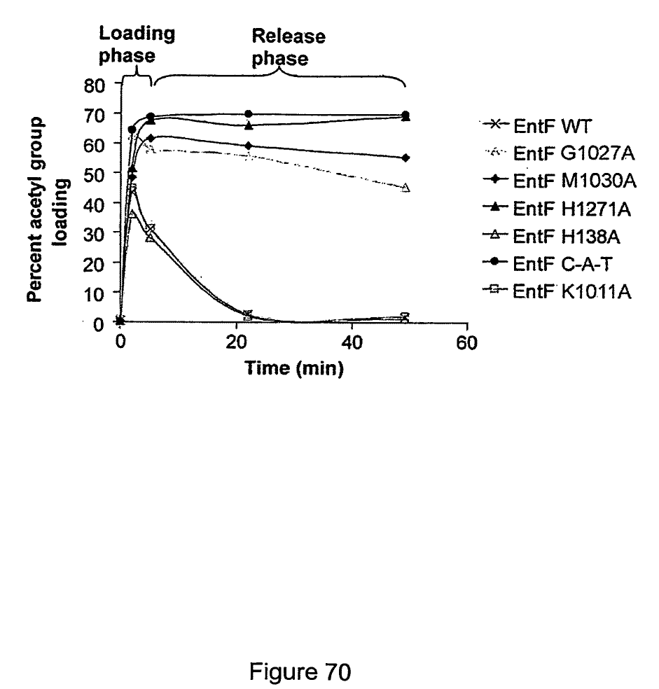

The EntF TE domain is a unique thioesterase because it is responsible for elongation (trimerization of DHB-Ser via the sidechain hydroxyl of Ser) followed by macrocyclization and release of the mature enterobactin product. This process requires well-timed communication events between the T and TE domains. From the enterobactin reconstitution assay, we concluded that the overall competence of the mutants G1027A and M1030A for the three steps involved in enterobactin production ([1] Ser loading, [2] condensation, and [3] elongation/macrocyclization) was reduced by 15- and 30-fold, respectively. However, neither of these mutations had defects in the Ser loading step or the condensation step as judged by assays that tested each of these steps separately. Therefore, we infer that G1027A and M1030A must be defective in the macrocyclization step (i.e., communication between the T and TE domains of EntF). As a direct assay for T-TE communication using the native DHB and Ser substrates is not available, we developed an assay to examine transfer of an independently primed acyl group from the T domain to the TE domain of EntF. In this assay, T-TE communication was detected by monitoring the net hydrolysis of a noncognate acyl group from EntF. In particular, a limiting amount of 1-[¹⁴C]-acetyl-CoA was used with Sfp to load the apo form of EntF (wt and mutants) with 1-[¹⁴C]-acetyl-pantethene onto the T domain. In wt EntF, this radiolabeled acyl group is transferred to the active site serine of the downstream TE domain but is not capable of participating in macrocyclization. As shown in FIG. 70, the 1 -[¹⁴C]-acetyl-O-TE was hydrolyzed to liberate [¹⁴C]-acetate. This phenomenon was manifested as an initial increase in the incorporation of radiolabel (loading catalyzed by Sfp) followed by a loss of the label over time (corresponding to hydrolysis of the radiolabeled acetyl group by the TE domain of EntF). The hydrolysis of acyl-O-TE intermediates is the default behavior of many TE domains in NRPS assembly lines. For the mutants G1027A and M1030A, the covalent label remains stably incorporated, consistent with a failure to be transferred to the TE domain for hydrolysis. Two additional types of controls were performed to further validate that this assay indeed examined the T-TE interaction, shown in FIG. 70. The first control utilized the C-A-T tridomain construct of EntF (with the wild-type sequence in the T domain). The 1-[¹⁴C-]-acetyl-S-T intermediate was stable for the wt tridomain, as expected if hydrolysis requires passage to the TE domain for its catalyzed hydrolysis activity. In the second control, an EntF mutant was assayed in which the histidine in the TE active site that acts as general base was altered (EntF H1271A). When primed with 1-[¹⁴C]-acetyl-pantetheinyl prosthetic group, the EntF TE domain mutant H1271A also did not show the time-dependent loss of radiolabel that was observed with wt EntF. These results indicate that the catalyzed loss of the radioactive acetyl group is dependent on a functional TE domain for EntF. Finally, mutations elsewhere in EntF, H138A in the C domain and K1011A in the T domain, expected to be functional in acyl transfer from T to TE domain, undergo acetyl group hydrolysis (FIG. 70). These results indicate that mutations elsewhere in EntF, which are not expected to affect T-TE domain communication, display behavior similar to wt in this assay. The loss of the acetyl radiolabel under these conditions thus provides insight into T-TE communication; and furthermore shows that the T-TE interaction is deficient in the EntF mutants G1027A and M1030A.

The T domains that are the centerpiece of the covalent attachment strategy for PKS and NRPS assembly line logic must first be primed by dedicated PPTases that add the 20 Å phosphopantetheine arm, thereby installing the nucleophilic thiol and bringing the assembly lines to the ready position. The thiols of the thiolation domains in turn capture acyl chains in covalent thioester linkage during natural product chain growth. The structure of a number of T domains, of both the ACP and PCP subcategories, have been determined by NMR and/or X-ray in both apo and holo forms and show a three- or four-helix scaffold with the Ser residue to be primed with phosphopantetheine near the N-terminal end of helix II. Priming by PPTase requires the folded architecture of the apoT domains for modification to proceed.

Despite the very similar folds among the 80-100 residue T domains, they can exist in several contexts. One major subgroup is that of free-standing T domains in type II PKS systems such as the actinorhodin, and the frenolicin synthases. At the other extreme are type I PKSs, such as deoxyerythronolide B synthase and rapamycin synthase, where a T domain is embedded in cis in every module. Most NRPS assembly lines follow type I assembly logic, e.g., ACV synthetase, tyrocidine synthetase, and the three subunit heptapeptide synthetase in vancomycin construction However, in coumermycin formation, there is a free standing A and T domain for channeling proline down that antibiotic pathway. The EntEBF synthetase is a hybrid of type I (EntF) and type II (EntBE) contexts with one T domain (EntB) in trans and one T domain (EntF) in cis.

Here, we have turned to the other T domain in the Ent synthetase, which is embedded within the four domain EntF and have used the same approach of shotgun alanine scanning and selection for survivors on low iron medium. We kept side chains of core residues in the EntF T domain constant and varied surface residues on helices II and III and in corresponding loops. The positions L1007, G1027, and M1030 could not be mutated to Ala without impaired enterobactin production. The L007A, G1027A, and M1030A mutants of EntF were constructed, purified, and assayed in vitro to validate the defect in Ent formation and to determine which of the domain-domain interactions was affected. First, the priming from apo-EntF to the holo form of the T domain still occurs in G1027A and M1030A but not L1007A. This assay provided a readout that the architecture of the T domain in the vicinity of the critical Ser to be primed is in a native state, and the results indicated that G1027A and M1030A were still competent in this regard, but L1007A was not. Second, the A domain within G1027A and M1030A still activates Ser and installs it on the holo form of the T domain as assayed by covalent loading of radiolabeled Ser onto EntF. The C domain was assayed in truncated three-domain C-A-T constructs of G1027A and M1030A with ¹⁴C-labeled Ser and unlabeled DHB with EntE and EntB. In the absence of a TE domain, if the C domain is functioning, it should transfer DHB from DHB-S-EntB to [¹⁴C]-Ser-S-EntF and yield the DHB-[¹⁴C]-Ser-S-EntF. Cleavage of the thioester allowed detection and quantitation of DHB-[¹⁴C]-Ser. Both the G1027A and M1030A forms of EntF were as active as wild-type EntF in this assay, suggesting recognition of the T domain mutants by the C domain in cis was unaffected.

With the C and A domains of EntF unaffected, the most likely effect of the G1027A and M1030A mutations in the EntF T domain are in its recognition by the in cis downstream TE domain. A result consistent with the impairment of T-TE interaction was obtained in an acyl transfer assay. EntF was primed with 1-[¹⁴C]-acetyl-CoA. Wild-type EntF hydrolyzes the acetyl thioester, presumably by transfer to the adjacent TE domain, which then acts as an acetyl-thioesterase. The half-life for acetyl group hydrolytic release is about 5 min. Compared to normal enterobactin cyclotrimerization of 100 min⁻¹, the hydrolysis of the noncognate acetyl group occurs at about 1/500th the rate, slow enough to be inconsequential for normal turnover but useful as an assay for a slow default hydrolytic activity of EntF TE domain. The G1027A and M1030A mutants in EntF can be stably primed with the acetyl-S-pantetheine consistent with failure to transfer the acetyl group from T to TE.

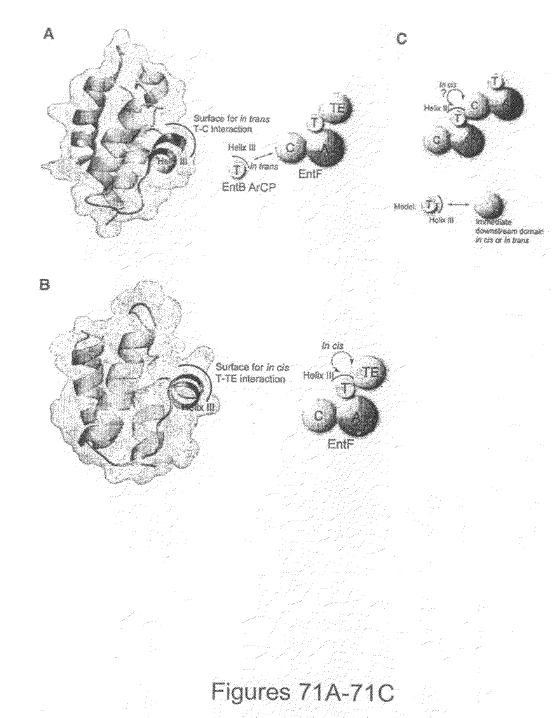

Both of the T domains in EntB and EntF have surface patches that are loci of specific recognition by particular partner enzymes. In the EntB T domain, two residues on helix III (F264 and A268) and one on helix II (M249) interact with the downstream EntF and are critical for C domain function (FIG. 71A). The EntF C domain is the immediate downstream catalytic domain that mediates DHB transfer from the EntB T domain scaffold. In the EntF T domain, the G1027 and M1030 are likewise on helix III and also are recognized by the immediate downstream catalytic domain, in this case the TE domain (FIG. 71B).

We believe that T domains use helix III as a general interaction surface for immediate downstream domains (FIG. 71C; TE domain in cis, C domain in cis or in trans). Structure analysis of the PCP from the third module of the tyrocidine synthetase TycC3-PCP has revealed the conformational motions that can mediate protein interactions in NRPS systems. Here, residues from helix III again were found to participate in domain-domain interaction. Other reports have also shown that helix III can be highly mobile in other carrier proteins, indicating that they may play roles in mediating protein interactions for these systems, too. In both EntB and EntF, the iron-dependent selection can be utilized to identify residues involved in slow catalytic steps. Structural studies (NMR, X-ray crystallography) can be sequenced to gain a complete understanding of domain-domain interactions in the enterobactin synthetase. Thiolation domains must be versatile to dock with distinct partner proteins, and the pantetheinyl arm can swivel over an arc of 120 degrees to populate distinct T domain conformers, movements that undoubtedly affect recognition by partner proteins. The conformational rearrangements in T domains may be analogous to mobile conformations of switch regions in G proteins that alter recognition by partner protein components. T domains may be workhorse scaffolds in natural product assembly lines where the pantetheinyl arm mobility, conformational dynamics, and surface residue recognition control growing chain flux through these way stations.

### Experimental Procedures

Production of a Homology Model for EntF T Domain

The T domain of EntF (residues 960-1047) was aligned with TycC3-PCP (PDB code: 1DNY) with the ClustalW algorithm. A homology model was generated by Swiss-Pdb Viewer and refined by SWISS-MODEL software. All structural figures were prepared with Pymol software (DeLano Scientific).

Library Construction and Selection for Enterobactin Production

For each library, an inactive template based on wild-type EntF construct pER311A was generated by the SOE method (Ho et al., Gene 77:51-59, 1989). The inactive templates contained tandem TAA stop codons followed by a unique restriction site SacI in the region of EntF T domain to be randomized. These inactive templates were used for full plasmid replication with the primers 5′-GCG CTT GGC GGT CAT TCG SYT SYT GCA RYG RMA CTG GCA SMA CAG TTA AGT CGG CAG GTT-3′ for helix U library, 5′-CGC CAG GTG ACG CCG GGG SMA GYT RYG GYT SMA TCA ACT GTC GCC AAA CTG-3′ for helix III library, and 5′-CAG TTA AGT CGG CAG GTT GCA SST SMA GYT RCT SCA GST CAA GTG ATG GTC GCG TCA-3′ for loop II/helix III library, respectively (sites of randomization indicated in bold; DNA degeneracies are represented as: K=G/T, M=A/C, R=AG, S=G/C, Y=C/T). DpnI and SacI were used to destroy the templates. Library DNA were transformed into electrocompetent entF::cat cells and plated onto minimal media in which iron was sequestered by the addition of 100 μM 2,2′-dipyridyl. The transformats were allowed to grow for 24 hr, and the largest colonies were isolated and sequenced.

Site-Directed Mutagenesis, Protein Expression, and Purification

The EntF site-directed mutants L1007A, K1011A, G1027A, and M1030A were constructed by the SOE method (Ho et al., supra). The generation of H1271A and H138A were previously described (Roche and Walsh, Biochemistry 42:1334-1344, 2003). The overexpression and purification of EntF (wild-type and mutants), EntE, EntB-ArCP, EntD, and Sfp were performed as reported (Roche and Walsh, supra). Protein concentrations were determined by Bradford assay.

Phosphopantetheinylation Assays

Phosphopantetheinylation was measured by incorporation of radiolabeled [³H]CoASH onto EntF (wt and mutants). Reactions were performed under the following condition: 75 mM Tris (pH 7.5), 10 mM MgCl₂, 0.5 mM Tris(2-Carboxyethyl) phosphine (TCEP), 6 μM EntF (wt and mutants), 30 μM [³H]CoASH (66.8 Ci/mol), and they were initiated by the addition of 1 μM EntD. Reactions were quenched with of 10% (wt/vol) TCA, and then BSA (100 mg) was added as a carrier. The protein pellet was washed with 10% (wt/vol) TCA and resuspended in formic acid, and the amount of radioactive label was measured by liquid scintillation counting.

Enterobactin Reconstitution Assay

Holo EntB-ArCP and EntF were prepared by incubating the apo proteins with 300 nM Sfp and 500 mM CoASH in 75 mM Tris (pH 7.5), 10 mMMgCl₂, and 0.5 mMTCEP for 20 min. The enterobactin reconstitution assay was performed as in [37] and modified to the following condition: 75 mM Tris (pH 7.5), 10 mM MgCl₂, 0.5 mM TCEP, 500 mM DHB, 1 mM L-serine, 10 mM ATP, 300 nM EntE, 15 mM holo EntB-ArCP, 100 nM holo EntF (wt or mutants). Reaction progress was monitored by high-performance liquid chromatography (HPLC) with water/acetonitrile/trifluoroacetic acid mobile phases. Duplicate experiments were performed to determine initial rates for enterobactin reconstitution.

Ser Incorporation Assay

Reactions were performed under the following condition: 75 mMTris (pH 7.5), 10 mM MgCl₂, 0.5 mM TCEP, 5 μM holo EntF (wt and mutants), 200 μM [¹⁴C] L-Ser (52.38 Ci/mol, Sigma), and they were initiated by the addition of 10 mMATP. The measurement of the amount of radioactive label on proteins was performed the same as that described in the Phosphopantetheinylation Assay section. Experiments were performed in duplicates.

Condensation Assay

Holo form EntF C-A-T (wt and mutants) proteins were prepared as above. The reaction mixture containing 75 mM Tris (pH 7.5), 10 mMMgCl₂, 0.5 mMTCEP, 5 μM holo EnF C-A-T (wt and mutants), 10 μM EntB-ArCP, 900 nM EntE, 100 μM [¹⁴C] L-Ser(52.38 Ci/mol, Sigma), and 10 mM ATP were preincubated for 5 min to allow Ser loading. The condensation reactions were started by adding 500 μM DHB. Reactions were quenched within 15 s and washed with 10% TCA. The protein pellets were resuspended in 100 μl 0.5 M KOH. After 10 min incubation at room temperature, which allows the release of Ser or DHB-Ser from proteins, 10 μl of 50% TFA (trifluoroacetic acid) was added to acidify the mixture. Precipitation was removed by centrifugation and supernatants were analyzed by HPLC. Flow-through radioactivity was monitored by using a Radioisotope Detector β-RAM Model 3 (Beckman).

Acyl Transfer Assay

Reactions were performed under this condition: 75 mMTris (pH 7.5), 10 mMMgCl₂, 0.5mMTCEP, 75 μM 1-[¹⁴C]acetyl-CoA (31.10 Ci/mol, Amersham Pharmacia), and 6 μM EnF (wt and mutants). Reactions were started by adding 300 nM Sfp. Reactions were quenched, and the amount of radioactive label was measured as described in the Phosphopantetheinylation Assay section.

### Other Embodiments

All publications, patent applications including U.S. provisional patent application No. 60/701,807, filed Jul. 21, 2005, and patents mentioned in this specification are herein incorporated by reference.

Various modifications and variations of the described method and system of the invention will be apparent to those skilled in the art without departing from the scope and spirit of the invention. Although the invention has been described in connection with specific desired embodiments, it should be understood that the invention as claimed should not be unduly limited to such specific embodiments. Indeed, various modifications of the described modes for carrying out the invention that are obvious to those skilled in the fields of medicine, immunology, pharmacology, oncology, or related fields are intended to be within the scope of the invention.

What is claimed is:

1. A method of generating a modified assembly line, said method comprising:
(a) providing a first gene encoding a polypeptide comprising at least one domain of a first assembly line;
(b) creating at least 15 unique mutations in the nucleic acid encoding said domain, thereby creating unique variants of said domain;
(c) introducing each of said variants into second genes encoding at least one domain of a second assembly line;
(d) expressing said second assembly line in a cell; and
(e) identifying a variant generated from step (b), using said cell in a selection or screen, wherein said selection or screen identifies a modified assembly line with an altered amount of a product or an altered structure of a product of said second assembly line, as compared to said unmodified second assembly line.

2. The method of claim 1, further comprising repeating steps (b) through (e) at least once recursively.

3. The method of claim 2, wherein said repeating is performed at least twice.

4. The method of claim 1, wherein said first assembly line and said second assembly line are derived from the same biosynthetic gene or genes.

5. The method of claim 1, wherein said first assembly line and said second assembly line are not derived from the same biosynthetic gene or genes.

6. The method of claim 1, wherein said first gene and said second gene are derived from the same biosynthetic gene.

7. The method of claim 1, further comprising replacing at least one domain in said second assembly line with a domain from a third assembly line prior to said identifying step (e).

8. The method of claim 7, wherein said third assembly line and said first assembly line are derived from the same biosynthetic assembly line.

9. The method of claim 1, wherein said polypeptide of step(a) comprises at least two domains of said first assembly line.

10. The method of claim 9, wherein step (b) creating further comprises modifying a second domain of said polypeptide coded for by said first gene.

11. The method of claim 1, wherein said step (b) comprises creating at least 25 variants.

12. The method of claim 11, wherein said step (b) comprises creating at least 50 variants.

13. The method of claim 12, wherein said step (b) comprises creating at least 100 variants.

14. The method of claim 13, wherein said step (b) comprises creating at least 500 variants.

15. The method of claim 14, wherein said step (b) comprises creating at least 1000 variants.

16. The method of claim 1, wherein said creating step (b) is performed in vitro.

17. The method of claim 1, wherein said creating step (b) is performed by random mutagenesis.

18. The method of claim 17, wherein said random mutagenesis is error prone PCR.

19. The method of claim 1, wherein said introducing step (c) comprises replacing at least one domain of said second gene with said variant.

20. The method of claim 1, wherein said cell is a bacterium.

21. The method of claim 20, wherein said bacterium is Bacillus subtilis, Pseudomonas syringae, Streptomyces sp., or Esherichia coli.

22. The method of claim 1, wherein said cell is a fungal cell.

23. The method of claim 22, wherein said fungal cell is a yeast cell.

24. The method of claim 1, wherein said selection or screen is performed by observing antibacterial or antifungal activity of said product.

25. The method of claim 1, wherein said selection or screen is performed on solid media.

26. The method of claim 1, wherein said selection or screen is performed in liquid media.

27. The method of claim 1, wherein said product is an antibiotic, antifungal, antineoplastic agent, or immunosupressant.

28. The method of claim 1, wherein said polypeptide comprises all domains of said assembly line.

29. The method of claim 1, wherein said first assembly line is an NRPS, a PKS, or an NRPS-PKS hybrid.

30. The method of claim 1, wherein said second assembly line is an NRPS, a PKS, or an NRPS-PKS hybrid.

31. An organism comprising a modified assembly line of claim 1.

32. A library produced by the method of claim 1, steps (a)-(c), said library comprising at least 15 nucleic acids encoding unique variants.

33. The library of claim 32, said library comprising at least 25 nucleic acids encoding unique variants.

34. The library of claim 33, said library comprising at least 50 nucleic acids encoding unique variants.

35. The library of claim 34, said library comprising at least 100 nucleic acids encoding unique variants.
